(19) United States
(12) Patent Application Publication
(10) Pub. No.: US 2016/0017540 A1
(43) Pub. Date: Jan. 21, 2016

(54) PRETREATMENT AND FRACTIONATION OF LIGNOCELLULOSIC BIOMASS

(71) Applicant: SUGANIT SYSTEMS, INC., Reston, VA (US)

(72) Inventors: Praveen PARIPATI, Reston, VA (US); Anantharam Prasad DADI, Toledo, OH (US)

(21) Appl. No.: 14/777,326

(22) PCT Filed: Mar. 14, 2014
(86) PCT No.: PCT/US14/29063

§ 371 Date: Sep. 15, 2015

(30) Foreign Application Priority Data
Mar. 15, 2013 (US) 13841707

(51) Int. Cl. D21C 3/02; C07D 307/12; D21C 9/02; C07C 59/185; D21C 3/20; C12P 19/02; C13K 1/02

(52) U.S. Cl. CPC D21C 3/02; C12P 19/02; C07D 307/12; C13K 1/02; C07C 59/185; D21C 3/20; D21C 9/02; C12P 2201/00

(57) ABSTRACT

Method and apparatus for enhanced production of sugars and lignin via fractionation of lignocellulosic biomass through ionic liquid pretreatment and mild alkaline treatment. The resulting biomass is easily fractionated and amenable to efficient and rapid enzymatic hydrolysis or acid hydrolysis and catalytic conversion to valuable products with high recovery of the enzymes used in the hydrolysis.

### CROSS-REFERENCE TO RELATED APPLICATIONS

This International Patent Application claims priority to U.S. patent application Ser. No. 13/841,707, filed Mar. 15, 2013, the disclosure of which is hereby incorporated by reference in its entirety.

### FIELD OF THE INVENTION

The present invention relates the sequential treatment of lignocellulosic biomass with ionic liquid pretreatment followed by mild alkaline treatment for efficient generation of cellulosic material and lignin fractions. The resulting cellulosic material may be efficiently and rapidly converted, by acid hydrolysis or enzymatic hydrolysis, to sugars, fuels, and chemicals and the lignin residue may be converted to chemicals and fuels.

### BACKGROUND OF THE INVENTION

Lignocellulose is the major structural component of plants and comprises cellulose, hemicellulose, and lignin. In lignocellulosic biomass, crystalline cellulose fibrils are embedded in a less well-organized hemicellulose matrix which, in turn, is surrounded by an outer lignin seal. lignocellulosic biomass is an attractive feed-stock because it is an abundant, domestic, renewable source that can be converted to liquid transportation fuels, chemicals and polymers. The major constituents of lignocellulose are: (1) hemicellulose (20-30%), an amorphous polymer of five and six carbon sugars; (2) lignin (5-30%), a highly cross-linked polymer of phenolic compounds; and (3) cellulose (30-40%), a highly crystalline polymer of cellobiose (a glucose dimer). Cellulose and hemicellulose, when hydrolyzed into their monomeric sugars, can be converted into ethanol fuel through well-established fermentation technologies. These sugars also form the feedstocks for production of a variety of chemicals and polymers. The lignin may also be recovered for use in the production of feedstock or used a fuel. The complex structure of biomass requires proper treatment to enable efficient hydrolysis (e.g., saccharification) of cellulose and hemicellulose components into their constituent sugars.

Practical means of producing chemicals and fuels from lignocellulosic biomass are limited due to the recalcitrant nature of cellulose in lignocellulosic biomass. Crystalline cellulose and hemicellulose are tightly sealed in the highly crosslinked lignin polymer, which acts as a physical barrier towards any chemical or biological attack on the carbohydrates in the biomass. Lignin is linked to carbohydrates via covalent and hydrogen bonds making biomass degradation difficult. Current treatment approaches suffer from slow reaction rates of cellulose hydrolysis (e.g., using the enzyme cellulase) and low sugar yields. Wyman, et al. (2005) Bioresource Technology 96: 1959-1966).

Contacting lingocellulosic biomass with hydrolyzing enzymes generally results in cellulose hydrolysis yields that are less than 20% of possible results. Hence, some “pretreatment” of the biomass may be carried out prior to attempting the enzymatic hydrolysis of the cellulose and hemicellulose in the biomass. Pretreatment refers to a process that converts lignocellulosic biomass from its native form, in which it is recalcitrant to cellulase enzyme systems, into a form for which cellulose hydrolysis is effective. Compared to untreated biomass, effectively pretreated lignocellulosic materials are characterized by an increased surface area (porosity) accessible to cellulase enzymes, and solubilization or redistribution of lignin. Increased porosity results mainly from a combination of disruption of cellulose crystallinity, hemicellulose disruption/solubilization, and lignin redistribution and/or solubilization. The relative effectiveness in accomplishing at least some of these factors differs greatly among different existing pretreatment processes.

The purpose of the pretreatment is to significantly disrupt the structure of biomass in order to: (a) reduce the crystallinity of cellulose, (b) increase accessibility/susceptibility of cellulose and hemicellulose chains to enzymes/catalysts by increasing the surface area/porosity and (c) remove lignin. U.S. Pat. No. 8,030,030. Several thermo-chemical biomass pretreatments techniques were investigated over the past few decades for improving the digestibility of this highly recalcitrant biomass. These include dilute acid, steam explosion, hydrothermal processes, “organosolv” processes involving organic solvents in an aqueous medium, ammonia fiber explosion (AFEX), strong alkali processes using a base (e.g., ammonia, NaOH or lime), and highly-concentrated phosphoric acid treatment. Many of these methods do not disrupt cellulose crystallinity, an attribute vital to achieving rapid cellulose digestibility. Also, some of these methods are not amenable for efficient recovery of the chemicals employed in the pretreatment.

In lime pretreatment, the biomass is pretreated with calcium hydroxide and water under different conditions of temperature and pressure. It can be conducted via (i) short-term pretreatment that lasts up to 6 h, requires temperatures of 100-160° C., and can be applied with or without oxygen (pressure ˜200 psig); and (ii) long-term pretreatment taking up to 8 weeks, requiring only 55-65° C., and capable of running with or without air (atmospheric pressure). Sierra, et al., Lime Pretreatment, in Biofuels, J. R. Mielenz, Editor. 2009, Humana Press. page 115-124. Alkali treatment of sugarcane bagasse at ambient conditions for up to 192 h followed by steam explosion at 200° C. at 1000 Psi for 5 minutes improved enzyme digestibility of the cellulose from 20% before pretreatment to 72% after pretreatment. Playne Biotechnology and Bioengineering, 1984. 26(5): 426-433.

Additionally, most alkali conditions were conducted at higher temperatures resulting in hemicellulose loss along with lignin removal. The Soda Process invented in 1851 uses 11 to 22% NaOH at 160-200° C. and 70 to 130 Psi for 4 to 5 hours to solubilize lignin which also results in hemicellulose removal. The Kraft process invented in 1884 uses combination of 15% NaOH and 5% NaS at 160-200° C. and 105-120 Psi for few hours to solubilize lignin which also removes hemicellulose. The Oxidative lime pretreatment was conducted with calcium hydroxide and oxygen at 150° C. and 200 Psi for 6 hours. During this treatment, about 38% of total biomass was solubilized, including 78% of lignin and 49% of xylan. Chang, et al., Oxidative lime pretreatment of high-lignin biomass. Applied Biochemistry and Biotechnology, 2001. 94(1): 1-28. Lime pretreatment of switch grass at 120° C. for 2 hours resulted in 10% glucan, 26% xylan and 29% lignin removal. Chang, et al., Lime pretreatment of switchgrass. Applied Biochemistry and Biotechnology, 1997. 63-65(1): 3-19.

Despite of many improvements in biomass pretreatment technologies, rapid production of monomeric sugars in high yields is still an area of active research. Majority of the pretreatments at cost effective strategies do not disrupt cellulose crystallinity, an attribute vital to achieving rapid hydrolysis rates. Therefore, enzymatic conversion of pretreated biomass from majority of these technologies still requires 48 to 168 hours to produce high yields. Other concentrated acid processes can enhance the hydrolysis rates, however, they are not practical to implement due to toxicity and recoverability issues. Therefore there exists a need in the art for a more efficient method of sugar and lignin generation from lignocellulosic biomass.

### SUMMARY OF THE INVENTION

In one embodiment, the method for the treatment of lignocellulosic biomass may comprise (a) mixing lignocellulosic biomass with an ionic liquid for a sufficient time and temperature to swell the lignocellulosic biomass without dissolution of the lignocellulosic biomass in the ionic liquid; and (b) treating the swelled lignocellulosic biomass under mild alkaline treatment to separate the lignin from the cellulose and hemicellulose.

In one embodiment, the method for the treatment of lignocellulosic biomass may comprise (a) mixing lignocellulosic biomass with an ionic liquid for a sufficient time and temperature to swell the lignocellulosic biomass without dissolving the lignocellulosic biomass in the ionic liquid; and (b) treating the swelled lignocellulosic biomass under mild alkaline treatment to separate the lignin from the cellulose and hemicellulose. In another embodiment, the lignocellulosic biomass may be washed after ionic liquid pretreatment in step (a) and before mild alkaline treatment in step (b). In another embodiment, the lignin may be recovered after the mild alkaline treatment. In a further embodiment, the recovered lignin may be processed to produce chemicals and fuels. In another embodiment, the cellulose and hemicellulose may undergo hydrolysis to produce sugars. In one embodiment, the hydrolysis is acid hydrolysis or enzymatic hydrolysis. In a further embodiment, the sugars may be further treated, preferably by acid hydrolysis, to produce chemicals, preferably chemicals are 5-hydroxymethyl furfural (HMF), furfural, 2,5-furandicarboxylic acid, formic acid, levulinic acid, or combinations thereof.

In one embodiment, a method for conversion of the carbohydrates of lignocellulosic biomass to sugars may comprise (a) mixing lignocellulosic biomass in an ionic liquid (IL) to swell but not dissolve the biomass; (b) applying radio frequency (RF) heating to the biomass to heat to a target temperature range; (c) applying ultrasonics, electromagnetic (EM), convective, conductive heating, or combinations thereof, to the lignocellulosic biomass to maintain the biomass at said target temperature range; (d) washing the treated lignocellulosic biomass; (e) subjecting said lignocellulosic biomass to mild alkaline treatment to release lignin from the cellulosic components; (f) washing the treated lignocellulosic biomass; and (g) hydrolyzing the treated cellulosic components, preferably cellulose and hemicellulose, to yield sugars. In one embodiment, the hydrolysis is acid hydrolysis or enzymatic hydrolysis. In a further embodiment, the sugars may be further treated, preferably by acid hydrolysis, to produce chemicals, preferably chemicals are 5-hydroxymethyl furfural (HMF), furfural, 2,5-furandicarboxylic acid, formic acid, levulinic acid, or combinations thereof. In another embodiment, the lignin may be recovered after the mild alkaline treatment. In a further embodiment, the recovered lignin may be processed to produce chemicals and fuels.

In one embodiment, a method for the conversion of cellulose to sugar may comprise (a) mixing lignocellulosic biomass in an ionic liquid (IL) to swell the lignocellulosic biomass; (b) applying radio frequency (RF) heating to the lignocellulosic biomass to heat to a target temperature range; (c) applying ultrasonics, electromagnetic (EM), convective, conductive heating, or combinations thereof, to the lignocellulosic biomass to maintain the biomass at said target temperature range; (d) washing the pretreated lignocellulosic biomass; (e) subjected said lignocellulosic biomass to mild alkaline treatment to release lignin from the cellulosic components; (f) washing the treated lignocellulosic biomass; and (g) hydrolyzing the treated cellulosic components to yield sugars and release lignin. In a further embodiment, the sugars may be further treated, preferably by acid hydrolysis, to produce chemicals, preferably chemicals are 5-hydroxymethyl furfural (HMF), furfural, 2,5-furandicarboxylic acid, formic acid, levulinic acid, or combinations thereof. In another embodiment, the lignin may be recovered after the mild alkaline treatment. In a further embodiment, the recovered lignin may be processed to produce chemicals and fuels.

In one embodiment, a method for treatment of lignocellulosic biomass may comprise (a) incubating a biomass in a sufficient amount of an ionic liquid (IL) for a sufficient time and temperature to swell the biomass without dissolution of the biomass in the IL; (b) applying radio frequency (RF) heating to the lignocellulosic biomass to heat to a target temperature range; (c) applying ultrasonic heating to the lignocellulosic biomass to maintain the biomass at said target temperature range; (d) washing the pretreated lignocellulosic biomass; (e) subjecting said lignocellulosic biomass to mild alkaline treatment to release lignin from the cellulosic components; (f) washing the treated lignocellulosic biomass with a liquid non-solvent for cellulose that is miscible with water and the IL; and (g) contacting said washed treated lignocellulosic biomass with an aqueous buffer may comprise enzymes capable of hydrolyzing cellulose and hemicellulose to produce sugars. In a further embodiment, the sugars may be further treated to produce chemicals, preferably chemicals are 5-hydroxymethyl furfural (HMF), furfural, 2,5-furandicarboxylic acid, formic acid, levulinic acid, or combinations thereof. In another embodiment, the lignin may be recovered after the mild alkaline treatment. In a further embodiment, the recovered lignin may be processed to produce chemicals and fuels.

In one embodiment, the biomass may be washed after step (a) and before step (b). In another embodiment, the lignin, cellulose, and/or hemicellulose may be recovered. In another embodiment, the method may further comprise processing the recovered lignin to produce chemicals, binders, plastics, fuels, or combinations thereof.

In another embodiment, the mild alkaline treatment may comprise the addition of an alkaline agent. In another embodiment, the alkaline agent may be NaOH, aqueous ammonia, LiOH, Mg(OH)₂, Al(OH)₃, Ca(OH)₂, H₂O₂, NaS, Na₂CO₃, or a combination thereof. In another embodiment, the alkaline agent may be added at a concentration of 1, 2, 3, 4, 5, 6, 7, 8, 9, 10, 11, 12, 13, 14, 15, 16, 17, 18, 19, 20, or 10-20% by weight. In another embodiment, the alkaline agent may be added at about 0.1, 0.2, 0.25, 0.3, 0.4, 0.5, 0.6, 0.7, 0.8, 0.9, 1, 2, 2.1, 2.2, 2.3, 2.4, 2.5, 3, 4, 5, 6, 7, 8, 8.25, 8.5, 8.75, 9, 10, 11, 12, 13, 14, 15, 16, 16.2, 16.4, 16.5, 16.65, 16.7, 16.8, 17, 18, 19, or 20% by weight. In another embodiment, the alkaline agent may be added at about 0.1-10%, 0.1-0.5%, 0.1-5%, 2-8%, 5-15%, 15-20%, 10-20% by weight.

In another embodiment, the mild alkaline condition comprise a pH of about 8-11, pH 8-10, pH 9-11, pH 9-10, pH 10-11, pH 9.5-10.5, 8, 8.25, 8.5, 8.75, 9, 9.25, 9.5, 9.75, 10, 10.25, 10.5, 10.75, or 11. In another embodiment, the pH may be about 8, 8.25, 8.5, 8.75, 9, 9.25, 9.5, 9.75, 10, 10.25, 10.5, 10.75, 11, 11.5, 12, 12.5 or 13.

In another embodiment, the alkaline treatment may be at a temperature of at least about 10° C., 20° C., 30° C., 40° C., 50° C., 60° C., 70° C., 40° C.-60° C., 50° C.-70° C., 50° C.-60° C., 40° C.-70° C., 40° C., 50° C., 70° C., 40° C.-60° C., 50° C.-70° C., 50° C.-60° C., or 40° C.-70° C. In another embodiment, the alkaline treatment may be at a temperature of about 10° C., 20° C., 30° C., 40° C., 50° C., 60° C., 70° C., 73° C., 75° C., 78° C., or 80° C. In another embodiment, the alkaline treatment may be at a temperature of about 10-50° C., 30-70° C., 40° C.-60° C., 50° C.-70° C., 50° C.-80° C., 40° C.-80° C., 50-80° C., 50° C.-70° C., 50° C.-60° C., or 40° C.-70° C.

In another embodiment, the mild alkaline treatment may be for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 minutes. In another embodiment, the mild alkaline treatment may be for about 1-60, 1-70, 1-75, 1-80, 1-30, 1-20, 5-10, or 1-15 minutes. In a further embodiment, the mild alkaline treatment may comprise about 0.5%, 1%, 2%, 3%, 4%, or 5% NaOH or KOH at about a pH of 9-10 for about 30 minutes at 40-50° C.

In one embodiment, the additional heating may comprise intermittent agitation during heating.

In one embodiment, the ionic liquid may be molten at a temperature ranging from about 10° C. to 160° C. and may comprise cations or anions. In another embodiment, the ionic liquid may comprise a cation structure that includes ammonium, sulfonium, phosphonium, lithium, imidazolium, pyridinium, picolinium, pyrrolidinium, thiazolium, triazolium, oxazolium, or combinations thereof. In another embodiment, the ionic liquid may comprise a cation selected from imidazolium, pyrrolidinium, pyridinium, phosphonium, ammonium, or a combination thereof. In another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the method further may comprise treating said treated biomass with a biochemical reagent to convert the cellulose and hemicellulose to sugars. In another embodiment, the sugars may be hexose and/or pentose sugars.

In another embodiment, the biochemical reagent may be an enzyme. In another embodiment, the biochemical reagent may be an enzyme mixture of hemicellulases, cellulases, endo-glucanases, exo-glucanases, and 1-β-glucosidases. In another embodiment, the cellulase may be cellobiohydrolase, endocellulase, exocellulase, cellobiase, endo-beta-1,4-glucanase, beta-1,4-glucanase, or mixtures thereof. In another embodiment, the hemicellulase may be laminarinase, lichenase, xylanase, or mixtures thereof. In another embodiment, the enzyme mixture further may comprise xylanases, arabinases, or mixtures thereof.

In one embodiment, the biochemical reagent may be a thermophilic enzyme. In another embodiment, the thermophilic enzyme may be active up to about 70° C. In another embodiment, the enzyme may be added at a concentration of about 0.5, 1, 2, 3, 4, or 5% by weight.

In one embodiment, the enzyme may be recovered. In another embodiment, at least 90, 91, 92, 93, 94, 95, 96, 97, 98, or 99% of the enzyme may be recovered.

In one embodiment, the enzyme may be reused. In a further embodiment, the enzyme may be reused for about 16-20 hydrolysis cycles. In a further embodiment, the enzyme may be reused for about 1, 2, 3, 4, 5, 6, 7, 8, 9, 10, 11, 12, 13, 14, 15, 16, 17, 18, 19, or 20 hydrolysis cycles. In a further embodiment, the enzyme may be reused for about 16-20 hydrolysis cycles.

In one embodiment, the biomass may be heated to about 51° C., 52° C., 53° C., 54° C., 55° C., 56° C., 57° C., 58° C., 59° C., 60° C. 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C. during enzyme hydrolysis.

In one embodiment, the method further may further comprise acid hydrolysis of the cellulose and hemicellulose to convert the cellulose and hemicellulose to sugars, chemicals, or combinations thereof. In another embodiment, the acid may be phosphoric acid, nitric acid, maleic acid, solid acids, sulfuric acid, hydrochloric acid, or a combination thereof. In another embodiment, the solid acid may be sulphamic acid, citric acid, oxalix acid, benzoic acid, CsHSO₄, CsHSeO₄, or a combination thereof. In another embodiment, the acid hydrolysis may be at a pH of about 1, 2, 3, 3.5, 4, 4.5, 5, 5.5, 5.8, 6, 6.5, 6.8, 1-3, 2-4, 3-5, 2-6, 3.5-4.5, or 4-6. In another embodiment, the acid may be added at acid concentration of at least about 0.01-1%, 0.1-2%, 0.1-4%, 0.2-4%, 0.05-5%, 0.01-5%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% by weight. In another embodiment, the acid hydrolysis may be at a temperature of at least about 80° C., 80° C.-200° C., 150° C.-180° C., 80° C.-240° C. 150° C.-160° C., 140° C.-170° C., 140° C., 150° C., 170° C., 180° C., 140° C.-160° C., 150° C.-170° C., 150° C.-180° C., or 140° C.-200° C. In another embodiment, the acid hydrolysis may be for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 110, 120, 130, 140, 150, 160, 170, 180, 190, 200, 210, 220, 230, 240, 250, 260, 270, 280, 290, 1-60, 1-80, 1-100, 1-120, 1-180, 1-200, 1-300, 1-340, or 1-360 minutes. In another embodiment, the acid hydrolysis may be at a pressure of at least about 100-1,000 kPa.

In one embodiment, the acid hydrolysis further comprises adding a catalyst. In another embodiment, the catalyst a metal halide, oxide, multifunctional homogenous catalyst, multifunctional heterogenous catalyst, resin, salt, zeolite, or a combination thereof. In another embodiment, the metal halide may be a metal fluoride, metal chloride, metal bromide, metal iodide, or metal astatide. In another embodiment, the zeolite may be analcime, chabazite, clinoptilolite, heulandite, natrolite, phillipsite, thromsonite, stilbite, gonnardite, natrolite, mesolite, paranatrolite, scolecite, tetranatrolite, edingtonite, kalborsite, analcime, leucite, pollucite, wairakite, Laumontite (LAU), yugawaralite (YUG), goosecreekite (GOO), montesommaite (MON), harmotome, phillipsite, amicite, gismondine, garronite, gobbinsite, or a synthetic zeolite, preferably Zeolite A. In another embodiment, the chemicals are renewable fuels, chemicals and materials, preferably ethanol, butanol, lactic acid, gasoline, biodiesel, methane, hydrogen, electricity, plastics, composites, protein, drugs, fertilizers, or combinations. In another embodiment, the chemicals are succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. In another embodiment, the chemicals are 5-hydroxymethyl furfural (HMF), furfural, 2,5-furandicarboxylic acid, formic acid, levulinic acid, or mixtures thereof.

In a further embodiment, the sugars may be converted to renewable fuels, chemicals and materials.

In a further embodiment, the heating may comprise at least two phases, a first phase may comprise application of electromagnetic (EM) heating, optionally a variable frequency in the electromagnetic spectrum, variable frequency heating, radiofrequency (RF) heating, or a combination thereof, and a second phase may comprise application of ultrasonics, electromagnetic (EM), convective, conductive heating, or combinations thereof.

In a further embodiment, the application of radiofrequency heating may be for about at least 5-10 seconds, 1-30 minutes, 5-30 minutes, or 20-240 minutes.

In a further embodiment, the application of ultrasonics, electromagnetic (EM), convective, conductive heating, or combinations thereof, may be for about at least 3-30 minutes, 5-30 minutes, or 3-4 hours.

In another embodiment, the method may further comprise washing the treated biomass. In yet another embodiment, the washing may comprise washing the biomass with a liquid non-solvent for cellulose that is miscible with water and the ionic liquid (IL). In another embodiment, the liquid non-solvent used for washing may be water, an alcohol, acetonitrile or a solvent which dissolves the IL. In yet another embodiment, the wash may be recovered and treated with RF heating to dehydrate the ionic liquid.

In yet another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

In one embodiment, the biomass may be agricultural residues including but not limited to corn stover, wheat straw, bagasse, rice hulls, or rice straw; wood and forest residues including but not limited to pine, poplar, douglas fir, oak, saw dust, paper/pulp waste, or wood fiber; kudzu; herbaceous energy crops including but not limited to switchgrass, reed canary grass, or miscanthus; lingocellulosic biomass including but not limited to may comprise lignin, cellulose, and hemicellulose; plant biomass, or mixtures thereof.

In another embodiment, the heating may comprise at least two phases, a first phase may comprise application of radiofrequency (RF) heating and a second phase may comprise application of ultrasonics, electromagnetic (EM), convective, conductive heating, or combinations thereof. In another embodiment, the application of radiofrequency heating may be for about at least 5-10 seconds, 1-30 minutes, 5-30 minutes, or 20-240 minutes. In another embodiment, the application of ultrasonics, electromagnetic (EM), convective, conductive heating, or combinations thereof may be for about at least 3-30 minutes, 5-30 minutes, or 3-4 hours. In another embodiment, the electromagnetic energy may be applied at a power of 100-1000 W, 1 KW-10 KW, or 5 KW-1 MW. In another embodiment, the radiofrequency may comprise a frequency between about 1-900 MHz, 300 kHz-3 MHz, 3-30 MHz, 30-300 MHz, 13, 13.56, 27, 27.12, 40, or 40.68 MHz. In another embodiment, the radiofrequency may penetrate the biomass to about 0.001 to 2.0 meters thickness. In another embodiment, the biomass may be heated to a temperature of at least about 1-300° C., 50° C.-100° C., 60° C.-130° C., 80° C.-175° C., or 100° C.-240° C. In another embodiment, the biomass may be treated with radiofrequency for at least about 1 minute to 100 hours, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

In one embodiment, the method may further comprise washing the treated biomass. In another embodiment, the washing may comprise washing the biomass with a liquid non-solvent for cellulose that is miscible with water and the ionic liquid (IL). In another embodiment, the liquid non-solvent used for washing may be water, an alcohol, acetonitrile or a solvent which dissolves the IL and thereby may extract the IL from the biomass. In another embodiment, the alcohol may be ethanol, methanol, butanol, propanol, or mixtures thereof.

In one embodiment, the ionic liquid may be recovered from the liquid non-solvent by a method selected from one or more of activated charcoal treatment, distillation, membrane separation, electro-chemical separation techniques, sold-phase extraction liquid-liquid extraction, or a combination thereof. In another embodiment, the ionic liquid may be recovered from the liquid non-solvent by application of electromagnetic heating including but not limited to radiofrequency heating, that dehydrates the ionic liquid. In another embodiment, the method may comprise the further step of reusing the recovered IL for treating more biomass including but not limited to wherein at least 90, 91, 92, 93, 94, 95, 96, 97, 98, or 99% of the IL may be recovered. In another embodiment, the ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%.

In one embodiment, the method may comprise incubating the biomass in a sufficient amount of an ionic liquid (IL) for a sufficient time to swell the biomass. In one embodiment, the method may comprise incubating the biomass in a sufficient amount of an ionic liquid (IL) for a sufficient time to swell the biomass but not dissolve the biomass. In one embodiment, the biomass may be not dissolved in the ionic liquid. In another embodiment, the incubating step may comprise incubating the biomass for a time ranging from about 5 minutes to about 8 hours. In another embodiment, the incubating step may comprise incubating the biomass at a temperature ranging from about 50° C. to about 200° C. In a further embodiment, the cellulose and hemicellulose structure in the biomass is swollen at least about 10%, 20%, 30%, or 40% by volume compared to before the ionic liquid incubation step.

In one embodiment, the biomass may be heated by heating with agitation, ultrasonics heating, electromagnetic (EM) heating, convective heating, conductive heating, microwave irradiation, or a combination thereof including but not limited to with intermittent agitation during heating.

In one embodiment, the ionic liquid may be molten at a temperature ranging from about 10° C. to 160° C. and may comprise cations or anions. In another embodiment, the ionic liquid may comprise a cation structure that includes ammonium, sulfonium, phosphonium, lithium, imidazolium, pyridinium, picolinium, pyrrolidinium, thiazolium, triazolium, oxazolium, or combinations thereof. In another embodiment, the ionic liquid may comprise a cation selected from imidazolium, pyrrolidinium, pyridinium, phosphonium, ammonium, or a combination thereof. In another embodiment, the ionic liquid (IL) may be 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof. In another embodiment, the ionic liquid may be 1-Butyl-3-methylimidazolium alkylbenzenesulfonate, 1-Ethyl-3-methylimidazolium alkylbenzenesulfonate, 1-Butyl-3-methylimidazolium acesulfamate, 1-Ethyl-3-methylimidazolium acesulfamate, 1-Ethylpyridinium chloride, 1-Butylimidazolium hydrogen sulfate, 1-Butyl-3-methylimidazolium hydrogen sulfate, 1-Butyl-3-methylimidazolium methyl sulfate, 1,3-Dimethylimidazolium methyl sulfate, 1-Butyl-3-methylimidazolium methanesulfonate, 1-Ethyl-3-methylimidazolium acetate, 1-Butyl-3-methylimidazolium acetate, 1-Octyl-3-methylimidazolium acetate, 1-(2-(2-Hydroxy-ethoxy)ethyl)-imidazolium acetate, 1-(2-(2-Methoxy-ethoxy)ethyl)-3-ethylimidazolium acetate, 1-(3,6,9,12-Tetraoxatridec-1-yl)-3-ethylimidazolium acetate, 1-(3,6,9,12,15,18,21-Heptaoxadocos-1-yl)-3-ethylimidazolium acetate, 1-(2-(2-Methoxy-ethoxy)ethyl)-triethylammonium acetate, (2-Hydroxy-ethyl)-dimethylammonium acetate, (2-Methoxyethyl)-dimethylammonium acetate, Tetramethylguanidinium acetate, Tetramethylguanidinium propionate, 1-Butyl-3-methylimidazolium formate, Tetrabutylphosphonium formate, Tetrabutylammonium formate, 1-Hexyl-3-methylimidazolium trifluoromethanesulfonate, 1-Butyl-3-methylimidazolium chloride, 1-Ethyl-3-methylimidazolium chloride, 1-Butyl-3-methylimidazolium bromide, 1-Allyl-3-methylimidazolium chloride, 1-Butyl-2,3-dimethylimidazolium tetrafluoroborate, 1-Butyl-3-methylimidazolium tetrafluoroborate, 1-Butyl-3-methylimidazolium hexafluorophosphate, 1-Butyl-1-methylpyrrolidinium hexafluorophosphate, 1-Ethyl-3-methylimidazolium diethyl phosphate, 1-Ethyl-3-methylimidazolium nitrate, 1,3-Dimethylimidazolium dimethyl phosphate, 1-Butyl-3-methylimidazolium dimethyl phosphate, 1-Methylimidazolium chloride, or combinations thereof.

In one embodiment, the IL may be represented by the structure:

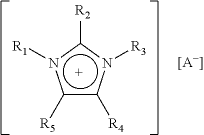

wherein each of R1, R2, R3, R4, and R5 is hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

In one embodiment, the IL may be represented by the structure:

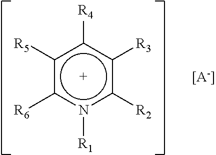

wherein each of R1, R2, R3, R4, R5, and R6 is hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

In one embodiment, the halide may a chloride, fluoride, bromide or iodide.

In one embodiment, the IL may be ionic liquid may be an ionic liquid mixture with a composition described by Equation 1:

$${\sum }_{n = 1}^{20} {{[ {C}^{+} ]}_{n} [ {A}^{-} ]}_{n} \qquad ( 1 )$$

C⁺ denotes the cation of the IL and A⁻ denotes the anionic component in Equation 1.

In one embodiment, the method may be a continuous process. In another embodiment, the method may be a batch process.

In one embodiment, the conditions of said biomass undergoing radiofrequency (RF) heating may be monitored by means of sensors including but not limited to a liquid flow rate sensor, thermocouple sensor, temperature sensor, salinity sensor, or combinations thereof. In another embodiment, the method may comprise adjusting the amount of ionic liquid, the time of incubation, or the temperature of the biomass.

In one embodiment, the method may further comprise treating said treated biomass with biochemical reagents including but not limited to an enzyme, to convert the cellulose and hemicellulose to sugars including but not limited to hexose and pentose sugars. In another embodiment, the biochemical reagent used to convert the cellulose and hemicellulose may be an enzyme including but not limited to an enzyme mixture of hemicellulases, cellulases, endo-glucanases, exo-glucanases, and 1-β-glucosidases. In another embodiment, the cellulase may be cellobiohydrolase, endocellulase, exocellulase, cellobiase, endo-beta-1,4-glucanase, beta-1,4-glucanase, or mixtures thereof.

In another embodiment, the hemicellulase may be laminarinase, lichenase, xylanase, or mixtures thereof. In another embodiment, the enzyme mixture may further comprise xylanases, arabinases, or mixtures thereof. In another embodiment, the biochemical reagents are thermophilic enzymes including but not limited to enzymes that are active up to about 70° C. In another embodiment, the biomass may be heated to at least about 50-100° C., 40° C., 55° C., or 70° C.

In another embodiment, the sugars may be converted to renewable fuels, chemicals and materials including but not limited to ethanol, butanol, lactic acid, gasoline, biodiesel, methane, hydrogen, electricity, plastics, composites, protein, drugs, fertilizers or other components thereof. In another embodiment, the chemicals may be succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

In one embodiment, the method may further comprise recovering the enzymes. In another embodiment, at least about 60, 70, 80, 90, 91, 92, 92, 93, 94, 95, 96, 97, 98, 99, or 100% of the enzymes may be recovered.

In one embodiment, the treatment produces a solid residue may comprise proteins, lignin, and/or ash. In another embodiment, the proteins and/or lignin may be recovered for use as feedstock or fuel. In a further embodiment, the lignin may be recovered.

In one embodiment, the method further may comprise treating said treated biomass with chemical reagents to convert the cellulose and hemicellulose to sugars including but not limited to hexose and pentose sugars. In another embodiment, the sugars may be converted, preferably by acid hydrolysis, to chemicals including but not limited to succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

In one embodiment, the reactor may be loaded with a high level of biomass. In another embodiment, the biomass may comprise high solids loadings at least about 10%, 20%, 30%, 40%, 50%, 60%, 70%, 80%, 90%, or 95% w/w. In a further embodiment, the biomass may be loaded at high solids loading at approximately 30% w/w.

In one embodiment, the biomass may be comminuted to smaller sized particles. In another embodiment, the biomass may be comminuted to smaller sized particles prior to mixing with an ionic liquid. In a further embodiment, the biomass may be comminuted to small particles about 0.1-20 mm, 0.1-2 mm, or about 5 mm in size.

In another embodiment, the treated biomass may be further processed to yield renewable fuels, chemicals and materials including but not limited to ethanol, butanol, lactic acid, gasoline, biodiesel, methane, hydrogen, electricity, plastics, composites, protein, drugs, fertilizers or other components thereof.

In one embodiment, the biomass may be subjected to additional heating with agitation, ultrasonic heating, electromagnetic (EM) heating, convective heating, conductive heating, microwave irradiation, or a combination thereof, preferably during step (a), step (b), acid hydrolysis, or enzymatic hydrolysis. In another embodiment, the additional heating may comprise intermittent agitation during heating. In another embodiment, the biomass may be heated by agitation, ultrasonic heating, electromagnetic (EM) heating, convective heating, conductive heating, microwave irradiation, or a combination thereof. In another embodiment, the electromagnetic (EM) heating may be radiofrequency (RF) heating or infrared (IR) heating. In another embodiment, the electromagnetic energy may be applied at a power of 100-1000 W, 1 KW-10 KW, or 5 KW-1 MW. In another embodiment, the radiofrequency comprises a frequency between about 1-900 MHz, 300 kHz-3 MHz, 3-30 MHz, 30-300 MHz, 13, 13.56, 27, 27.12, 40, or 40.68 MHz. In another embodiment, the infrared radiation may be at a frequency range of about 430 THz down to 300 GHz. In another embodiment, the infrared radiation may be near-infrared (near IR) wavelengths at about 0.75-1.4 μm, mid-infrared (mid IR) wavelengths at about 3-8 μm, or far infrared (far IR) wavelengths at about 15-1,000 μm. In another embodiment, the radiofrequency heating may penetrate the biomass to about 0.001 to 2.0 meters thickness. In another embodiment, the infrared heating penetrates the biomass to about 0.001 to 2.0 meters thickness. In another embodiment, the heating comprises at least two phases, a first phase comprising application of electromagnetic (EM) heating, preferably a variable frequency in the electromagnetic spectrum, variable frequency heating, infrared (IR) heating, variable (IR) heating, radiofrequency (RF) heating, or a combination thereof, and a second phase comprising application of ultrasonics, electromagnetic (EM), convective, conductive heating, or combinations thereof.

In one embodiment, the biomass may be heated to a temperature of at least about 50-200° C., 80° C.-240° C., 50° C.-100° C., 60° C.-130° C., 80° C.-175° C., 100° C.-240° C., 90° C., 100° C., 105° C., 110° C., 115° C., 120° C., 125° C., 130° C., 135° C., 140° C., 145° C., or 150° C. In another embodiment, the biomass may be heated for at least about 1 minute to 100 hours, 5 minutes to 8 hours, 3-30 minutes, 5-30 minutes, 3-4 hours, least 5-10 seconds, 1-30 minutes, 5-30 minutes, 1-360 minutes, 20-240 minutes, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

In one embodiment, the biomass may be subjected to additional heating with agitation, ultrasonics heating, electromagnetic (EM) heating, preferably radiofrequency heating or infrared heating, convective heating, conductive heating, microwave irradiation, or a combination thereof, preferably with intermittent agitation during heating.

In one embodiment, the biomass may be agricultural residues, preferably corn stover, wheat straw, bagasse, rice hulls, or rice straw; wood and forest residues, preferably pine, poplar, douglas fir, oak, saw dust, paper/pulp waste, or wood fiber; algae; kudzu; coal; cellulose, lignin, herbaceous energy crops, preferably switchgrass, reed canary grass, or miscanthus; lingocellulosic biomass, preferably comprising lignin, cellulose, and hemicellulose; plant biomass; or mixtures thereof. In another embodiment, the lignocellulosic biomass may be agricultural residue, wood and forest residue, kudzu, herbaceous energy crop, lingocellulosic biomass comprising lignin, cellulose, and hemicellulose, plant biomass, or mixtures thereof.

In one embodiment, the method may be a continuous process. In another embodiment, the method may be a batch process. In another embodiment, the method may be a fed-batch process.

In one embodiment, the method may comprise adjusting the amount of ionic liquid, the time of incubation, the pH of the biomass, and the temperature of the biomass.

In one embodiment, the conditions of said biomass undergoing treatment may be monitored with sensors, preferably a liquid flow rate sensor, thermocouple sensor, temperature sensor, salinity sensor, or combinations thereof.

In one embodiment, the hemicellulose, cellulose, and/or lignin may be separated.

In one embodiment, the hemicellulose, cellulose, and/or lignin are converted to fuels, chemicals, polymers, or mixtures thereof.

In one embodiment, the method does not comprise the use of a cellulase or hemicellulase.

In one embodiment, the a method for conversion of the carbohydrates of lignocellulosic biomass to sugars may comprise mixing lignocellulosic biomass in an ionic liquid (IL) to swell but not dissolve the biomass; applying radio frequency (RF) heating to the lignocellulosic biomass to heat to a target temperature range; applying ultrasonics, electromagnetic (EM), convective, conductive heating, or combinations thereof, to the lignocellulosic biomass to maintain the lignocellulosic biomass at said target temperature range of about 50-220° C.; washing the treated lignocellulosic biomass; subjecting said lignocellulosic biomass to mild alkaline treatment to release lignin from the cellulosic components; washing the treated lignocellulosic biomass; recovering the lignin, cellulose, and hemicellulose; and hydrolyzing the cellulose and hemicellulose to yield sugars. In another embodiment, the hydrolyzing may be acid hydrolysis or enzyme hydrolysis.

In one embodiment, a method for treatment of lignocellulosic biomass may comprise incubating a biomass in a sufficient amount of an ionic liquid (IL) for a sufficient time and temperature to swell the lignocellulosic biomass without dissolution of the lignocellulosic biomass in the IL; applying radio frequency (RF) heating to the lignocellulosic biomass to heat to a target temperature range; applying ultrasonic heating to the lignocellulosic biomass to maintain the biomass at said target temperature range; washing the pretreated lignocellulosic biomass; subjecting said lignocellulosic biomass to mild alkaline treatment to release lignin from the cellulosic components; washing the treated lignocellulosic biomass with a liquid non-solvent for cellulose that is miscible with water and the IL; recovering the lignin, cellulose, and hemicellulose; and contacting said washed treated lignocellulosic biomass with an aqueous buffer comprising enzymes capable of hydrolyzing cellulose and hemicellulose to produce sugars. In one embodiment, the method of acidic hydrolysis of biomass may comprise reducing the biomass in size, preferably to particles about 0.1-20 mm in size; ionic liquid (IL) treatment of said biomass; treating the biomass uder mild alkaline treatment to separate the lignin from the cellulose and hemicellulose; separating the cellulosic, hemicellulosic, and lignin streams; recovering the lignin; adding an acid to each of the cellulosic and hemicellulosic streams to lower the pH below pH 7, preferably adding a catalyst; heating the cellulosic and hemicellulosic streams to heat to a target temperature range, preferably about 150-200° C. for about 15-360 minutes; and recovering chemicals.

In one embodiment, a method for conversion of the carbohydrates of lignocellulosic biomass to sugars may comprise mixing lignocellulosic biomass in an ionic liquid (IL) to swell but not dissolve the biomass; applying radio frequency (RF) heating to the lignocellulosic biomass to heat to a target temperature range; applying ultrasonics, electromagnetic (EM), convective, conductive heating, or combinations thereof, to the lignocellulosic biomass to maintain the lignocellulosic biomass at said target temperature range of about 50-220° C.; washing the treated lignocellulosic biomass; subjecting said lignocellulosic biomass to mild alkaline treatment to release lignin from the cellulosic components; washing the treated lignocellulosic biomass; recovering the lignin, cellulose, and hemicellulose; and hydrolyzing the cellulose and hemicellulose to yield sugars. In another embodiment, the hydrolyzing may comprise acid hydrolysis or enzyme hydrolysis.

In one embodiment, the method for treatment of lignocellulosic biomass may comprise (a) incubating a biomass in a sufficient amount of an ionic liquid (IL) for a sufficient time and temperature to swell the lignocellulosic biomass without dissolution of the lignocellulosic biomass in the IL; (b) applying radio frequency (RF) heating to the lignocellulosic biomass to heat to a target temperature range; (c) applying ultrasonic heating to the lignocellulosic biomass to maintain the biomass at said target temperature range; (d) washing the pretreated lignocellulosic biomass; (e) subjecting said lignocellulosic biomass to mild alkaline treatment to release lignin from the cellulosic components; (f) washing the treated lignocellulosic biomass with a liquid non-solvent for cellulose that is miscible with water and the IL; (g) recovering the lignin, cellulose, and hemicellulose; and (h) contacting said washed treated lignocellulosic biomass with an aqueous buffer may comprise enzymes capable of hydrolyzing cellulose and hemicellulose to produce sugars.

In one embodiment, the method of acidic hydrolysis of biomass may comprise reducing the biomass in size, preferably to particles about 0.1-20 mm in size; incubating a biomass in a sufficient amount of an ionic liquid (IL) for a sufficient time and temperature to swell the lignocellulosic biomass without dissolution of the lignocellulosic biomass in the IL; treating the biomass uder mild alkaline treatment to separate the lignin from the cellulose and hemicellulose; separating the cellulosic, hemicellulosic, and lignin streams; recovering the lignin; adding an acid to each of the cellulosic and hemicellulosic streams to lower the pH below pH 7, preferably adding a catalyst; heating the cellulosic and hemicellulosic streams to heat to a target temperature range, preferably about 150-200° C. for about 15-360 minutes; and recovering chemicals.

In one embodiment, the method for conversion of the carbohydrates of lignocellulosic biomass to sugars may comprise mixing lignocellulosic biomass in an ionic liquid (IL) to swell but not dissolve the biomass; applying ultrasonics, electromagnetic (EM), preferably radio frequency (RF), convective, conductive heating, or combinations thereof, to the lignocellulosic biomass to heat the lignocellulosic biomass at a temperature range of about 50-220° C.; ashing the treated lignocellulosic biomass; subjecting said lignocellulosic biomass to mild alkaline treatment to release lignin from the cellulosic components; washing the treated lignocellulosic biomass; recovering the lignin, cellulose, and hemicellulose; separating the lignin, cellulose, and hemicellulose; hydrolyzing the cellulose to yield sugars; and hydrolyzing the hemicellulose to yield sugars.

In one embodiment, the method for treatment of lignocellulosic biomass may comprise (a) incubating a biomass in a sufficient amount of an ionic liquid (IL) for a sufficient time and temperature to swell the lignocellulosic biomass without dissolution of the lignocellulosic biomass in the IL; (b) applying radio frequency (RF) heating, ultrasonic heating, or a combination to the lignocellulosic biomass to maintain at a target temperature range; (c) washing the pretreated lignocellulosic biomass; (d) subjecting said lignocellulosic biomass to mild alkaline treatment to release lignin from the cellulosic components; (e) washing the treated lignocellulosic biomass with a liquid non-solvent for cellulose that is miscible with water and the IL; (f) recovering the lignin, cellulose, and hemicellulose; (g) separating the lignin, cellulose, and hemicellulose; (h) hydrolyzing the cellulose to yield sugars; and (i) hydrolyzing the hemicellulose to yield sugars. In another embodiment, the hydrolyzing may comprise acid hydrolysis or enzyme hydrolysis. In another embodiment, the sugars may be processed by acid hydrolysis, preferably with a catalyst, to produce chemicals.

In one embodiment, the method of acidic hydrolysis of biomass may comprise reducing the biomass in size, preferably to particles about 0.1-20 mm in size; incubating a biomass in a sufficient amount of an ionic liquid (IL) for a sufficient time and temperature to swell the lignocellulosic biomass without dissolution of the lignocellulosic biomass in the IL; treating the biomass uder mild alkaline treatment to separate the lignin from the cellulose and hemicellulose; separating the cellulosic, hemicellulosic, and lignin streams; recovering the lignin; adding an acid to the cellulosic stream to lower the pH below pH 7, preferably adding a catalyst; adding an acid to the hemicellulosic stream to lower the pH below pH 7, preferably adding a catalyst; heating the cellulosic stream to heat to a target temperature range, preferably about 150-200° C. for about 15-360 minutes; heating the hemicellulosic stream to heat to a target temperature range, preferably about 150-200° C. for about 15-360 minutes; and recovering chemicals.

In one embodiment, the method of acidic hydrolysis of biomass may comprise reducing the biomass in size, preferably to particles about 0.1-20 mm in size; incubating a biomass in a sufficient amount of an ionic liquid (IL) for a sufficient time and temperature to swell the lignocellulosic biomass without dissolution of the lignocellulosic biomass in the IL; separating the cellulosic, hemicellulosic, and lignin streams; recovering the lignin; adding an acid to the cellulosic stream to lower the pH below pH 7, preferably adding a catalyst; adding an acid to the hemicellulosic stream to lower the pH below pH 7, preferably adding a catalyst; heating the cellulosic stream to heat to a target temperature range, preferably about 150-200° C. for about 15-360 minutes; heating the hemicellulosic stream to heat to a target temperature range, preferably about 150-200° C. for about 15-360 minutes; and recovering chemicals. In another embodiment, the chemicals may be succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. In another embodiment, the chemicals may be 5-hydroxymethyl furfural (HMF), furfural, 2,5-furandicarboxylic acid, formic acid, levulinic acid, or mixtures thereof.

### BRIEF DESCRIPTION OF THE DRAWINGS

FIG. 1 depicts a schematic of the pretreatment on lignocellulosic biomass. (A) the general structure of lignocellulosic biomass, (B) pretreatment depicting partial breakdown, and (C) improved pretreatment depicting complete disruption of the biomass structure including the elimination of cellulose crystallinity.

FIG. 2A depicts an exemplary method for processing biomass comprising mixing with ionic liquid to swell but not dissolve the biomass in the IL (e.g., for about 5 minutes to 8 hours) followed by mild alkaline treatment at about pH 8-11, optionally heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeating heating, washing the biomass, optionally recovering the IL. The mild alkaline treatment produces two outputs, a hydrolystate stream comprising cellulose and hemicellulose and a residue comprising lignin. The lignin may be recovered. The hydrolystate stream comprising cellulose and hemicellulose may be directed to further processing by the addition of cellulase and hemicellulases to their constituent monomeric sugars (e.g., five and six carbon sugars), optionally recovery of the added enzymes, to produce chemicals or biofuels. The residual solids (e.g., proteins and/or lignin) may be directed to further processing to produce feedstock or biofuels. The ionic liquid and enzymes may be reclaimed and reused.

FIG. 2B depicts an exemplary method for processing biomass comprising mixing with ionic liquid to swell but not dissolve the biomass in the IL (e.g., for about 5 minutes to 8 hours) followed by mild alkaline treatment at about pH 8-11, optionally heating by radio frequency irradiation to reach a target temperature range, optionally repeated, maintaining the temperature of the biomass using of ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeated, washing the biomass, optionally recovering the IL and dehydrating the IL by application of radiofrequency heating. The mild alkaline treatment produces two outputs, a hydrolystate stream comprising cellulose and hemicellulose and a residue comprising lignin. The lignin may be recovered. The hydrolystate stream comprising cellulose and hemicellulose may be directed to further processing by the addition of cellulase and hemicellulases to their constituent monomeric sugars (e.g., five and six carbon sugars), optionally recovery of the added enzymes, to produce chemicals or biofuels. The residual solids (e.g., proteins and/or lignin) may be directed to further processing to produce feedstock or biofuels. The ionic liquid and enzymes may be reclaimed and reused.

FIG. 2C depicts an exemplary method for processing biomass comprising mixing with ionic liquid to swell but not dissolve the biomass in the IL (e.g., for about 5 minutes to 8 hours) followed by mild alkaline treatment at about pH 8-11, optionally heating by radio frequency irradiation to reach a target temperature range, optionally repeated, maintaining the temperature of the biomass using of ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, optionally about 5-30 minutes, optionally repeated, washing the biomass, optionally recovering the IL and dehydrating the IL by application of radiofrequency heating. The mild alkaline treatment produces two outputs, a hydrolystate stream comprising cellulose and hemicellulose and a residue comprising lignin. The lignin may be recovered and undergo further processing to yield chemicals, fuels, and feedstocks. The hydrolystate stream comprising cellulose and hemicellulose may be directed to further processing by the addition of acids to their constituent monomeric sugars (e.g., five and six carbon sugars), and, optionally the sugars may undergo acid hydrolysis to produce chemicals or biofuels (e.g., HMF, furfural, levulinic acid). The acid hydrolysis may be performed in the presence of a catalyst.

FIG. 3 depicts exemplary cation and anion components of ionic liquids.

FIG. 4 depicts the percentage of glucan and xylan conversion to monomeric sugars. (A) pretreated (PT) wheatstraw plus alkaline treatment (CT) at 75° C. for 60 minutes; (C) pretreated (PT) wheatstraw with no alkaline treatment (CT); (C) no pretreatment (UT, untreated) wheatstraw plus alkaline treatment at 75° C. for 60 minutes, and (D) untreated wheatstraw.

FIG. 5 depicts an exemplary method for processing biomass comprising preparing the biomass (e.g., reducing to a smaller sized), conditioning the biomass (e.g., ionic liquid treatment, mild alkaline treatment, or combinations thereof), optionally heated or repeated, and where three stream are separated: cellulose, hemicellulose, and lignin. Each individual stream, cellulose, hemicellulose, and lignin then undergoes catalytic conversion. Catalytic conversion process reduces the cellulose, hemicellulose, and lignin to produce chemicals or biofuels. Resulting hemicellulosic sugar stream is separated from residual biomass substrates consisting mainly of cellulose and lignin. Hemicellulosic sugars thus obtained are further catalyzed to yield furfural or isomerized to produce xylulose. Remaining residual biomass consisting of cellulose and lignin is subjected to delignification using mild caustic treatment at conditions sufficient to enable removal of major lignin protions. Lignin in the alkali stream is separated and subjected to base catalyzed reactions for production of aromatic chemicals and low molecular weight lignin polymers for material applications. Residual biomass consisting mainly of cellulose substrates is treated with ionic liquid for sufficient time to remove crystallinity of cellulose for efficient enzymatic/acidic hydrolysis or efficient catalysis.

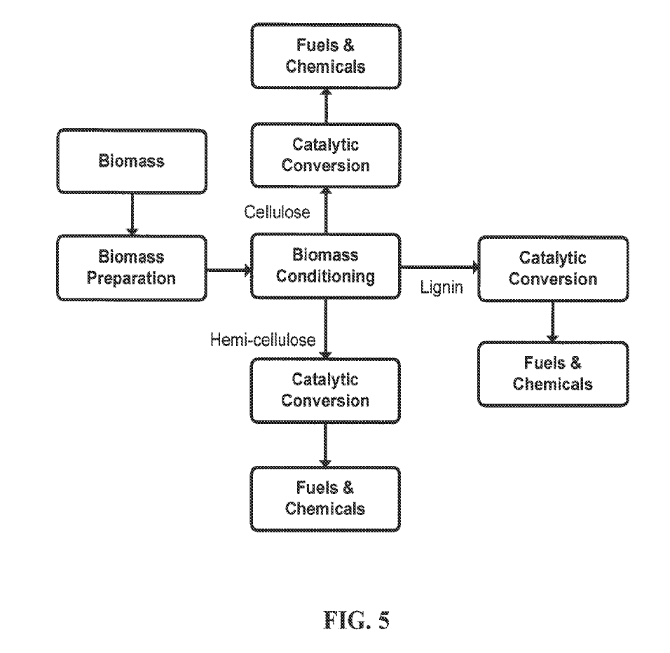

### DETAILED DESCRIPTION OF THE PREFERRED EMBODIMENTS

In order that the invention herein described may be fully understood, the following detailed description is set forth. Various embodiments of the invention are described in detail and may be further illustrated by the provided examples. Additional viable variations of the embodiments can easily be envisioned.

### Definitions

Unless defined otherwise, all technical and scientific terms used herein have the same meaning as those commonly understood by one of ordinary skill in the art to which this invention belongs.

As used in the description herein and throughout the claims that follow, the meaning of “a,” “an,” and “the” includes plural reference unless the context clearly dictates otherwise.

“Acid hydrolysis,” as used herein, refers broadly to a process of treating biomass with an acid (e.g., sulfuric acid, HCl, phosphoric acid, nitric acid, maleic acid, or solid acids) at low concentration (e.g., 1-4% by weight) for a brief time (e.g., about 1-360 minutes) at a lower temperature (e.g., 80-200° C.) and lower pressure (e.g., 100 kPa to 10×10⁵ kPa) at about pH 1-6 to convert cellulosic matter (e.g., cellulose and hemicellulose) into their constituent sugars and the sugars into chemicals (e.g. hydroxymethylfurfural (HMF) and furfural). This is in contrast to prior methods where the agents were used at high concentration, high temperature, and high pressure for longer periods of time (e.g., 30% at over 300° C. for 3-4 weeks at pH>11).

“Biomass,” as used herein, refers broadly to any biological material. Biomass encompasses substrates containing organic components which can be used in production of renewable fuels, chemicals and materials such as ethanol, butanol, lactic acid, gasoline, biodiesel, methane, hydrogen, plastics, composites, protein, drugs, fertilizers or other components thereof. Biomass may be agricultural residues including but not limited to corn stover, wheat straw, bagasse, rice hulls, or rice straw; wood and forest residues including but not limited to pine, poplar, Douglas fir, oak, saw dust, paper/pulp waste, or wood fiber; feedstock (e.g., woody biomass and agricultural biomass); kudzu; algae including but not limited to red algae; herbaceous energy crops including but not limited to switchgrass, reed canary grass, or miscanthus; lingocellulosic biomass; plant biomass; or mixtures thereof. Biomass may be lignocellulosic biomass comprising cellulose, hemicellulose, and lignin.

“Ionic liquids” as used herein, refers broadly to room temperature liquids that contain only ions and are molten salts stable up to 300° C. Sheldon (2001) Chem. Commun. 23: 2399-2407.

“lignocellulosic biomass” as used herein, refers broadly to plant biomass that is composed of cellulose, hemicellulose, and lignin. The carbohydrate polymers (e.g., cellulose and hemicelluloses) are tightly bound to the lignin. lignocellulosic biomass can be grouped into four main categories: agricultural residues (e.g., corn stover and sugarcane bagasse), dedicated energy crops, wood residues (e.g., sawmill and paper mill discards), and municipal paper waste.

“Mild alkaline treatment,” as used herein, refers broadly to a process of treating biomass with an alkaline agent (e.g., Ca(OH)₂, Mg(OH)₂, Al(OH)₃, NaOH) at low concentration (e.g., 1-5%) for a brief time (e.g., about 30 minutes) at a lower temperature (e.g., 40-70° C.) at about pH 8-13. This is in contrast to prior methods where the agents were used at high concentration, high temperature, for longer periods of time (e.g., 30% at 120-150° C. for 3-4 weeks at pH>11).

“Pretreatment of biomass,” as used herein, refers broadly to a process of changing the physiochemical structure of biomass to make it amenable for efficient conversion to their monomeric valuable products.

“Radiofrequency (RF) heating,” as used herein, refers broadly to application of electromagnetic field to biomass/products/dielectric materials at frequencies from about 1-300 MHz.

“Electromagnetic energy (EM),” as used herein, refers broadly to a form of energy that is reflected or emitted from objects in the form of electrical and magnetic waves that can travel through space. There are many forms of electromagnetic energy including gamma rays, x rays, ultraviolet radiation, visible light, infrared radiation, microwaves, and radio waves (radiofrequency).

“Ultrasonics” or “ultrasonic waves,” as used herein, refers broadly to sound waves (mechanical waves) with high frequency about between 15 kHz to 40 kHz (e.g., about 20 kHz) and low amplitude about between 0.0001-0.025 mm.

### Biomass Treatment Combination of IL Pretreatment and Mild Alkaline Treatment

The present invention provides a method for the treatment of biomass to yield useful chemicals comprising the combination of ionic liquid pretreatment followed by mild alkaline treatment.

This invention provides an efficient biomass disruption/fractionation strategy employing sequential ionic liquid pretreatment followed by mild alkaline treatment process which (a) can be used for treating any lignocellulosic biomass substrates, (b) results in efficient cellulosic material and lignin fraction generation at mild conditions (of temperature, pressure, time, chemical, solvents) enabling catalytic conversions of all constituents of biomass in a single or sequential steps (c) results in a de-crystalized and swollen cellulose for catalytic conversion to monomeric sugars or chemicals, (d) results in enhanced production of monomeric sugars, (e) results in a catalytically convertible lignin fraction for generation of aromatic chemicals, (f) results in purer fractions of cellulosic pulp and lignin fractions, (g) results in lower reagent, chemical and catalyst requirements, (h) enables economic recovery of catalysts and chemicals. The inventors surprisingly discovered that the combination of ionic liquid pretreatment with mild alkaline treatment allows for improved treatment of biomass and expectantly resulted in higher enzyme recovery levels (e.g., >90%) and greater enzyme longevity (e.g., 16-20 uses versus 1 use). Further, the cellulose and hemicellulose may be treated by acid hydrolysis for conversion into sugars. The sugars may also be treated by acid hydrolysis for conversion into chemicals (e.g., hydroxymethylfurfural (HMF) and furfural), optionally with a catalyst.

### Ionic Liquid (IL)

Ionic liquids are liquids at room temperature and may contain only ions and are molten salts stable up to 300° C. See Sheldon (2001) Chem. Commun. 23: 2399-2407. They contain cations which are usually organic compounds and anions of inorganic or organic components such that the resulting salts are asymmetric. Because of poor packing associated with the asymmetric nature of ILs, crystal formation is inhibited and ILs remain liquids over a wide range of temperatures. A wide range of anions and cations can be employed to generate ILs with varied melting points, viscosities, thermal stabilities and polarities. Examples of some of the cations currently used include ammonium, sulfonium, phosphonium, lithium, imidazolium, pyridinium, picolinium, pyrrolidinium, thiazolium, triazolium oxazolium, or combinations thereof. Murugesan & Linhardt (2005) Current Organic Synthesis 2: 437-451. Ionic liquids are also liquid at <100° C., broad liquid range, almost no vapor pressure, high polarity, high dissolving power for organic and inorganic materials, good thermal, mechanical, and electrochemical stability, high heat capacity, non-flammable, and electrical conductivity.

Ionic liquids have extremely low volatility and when used as solvents, they do not contribute to emission of volatile components. Ionic liquids are considered environmentally benign solvents. ILs have been designed to dissolve cellulose and lignocellulose.

The ionic liquid treatment differs from the classic approach to the use of ionic liquids in that the aim is not to dissolve lignocellulose, but rather to contact it with the IL for times sufficient to mainly disrupt lignin sheathing and swell the remaining biomass structure significantly (at least 30%) but not dissolve the lignocellulose. This combination treatment enables the subsequent enzymatic hydrolysis process to proceed in a relatively short period of time as well as give quantitative yields of glucose and high yields of pentose sugars.

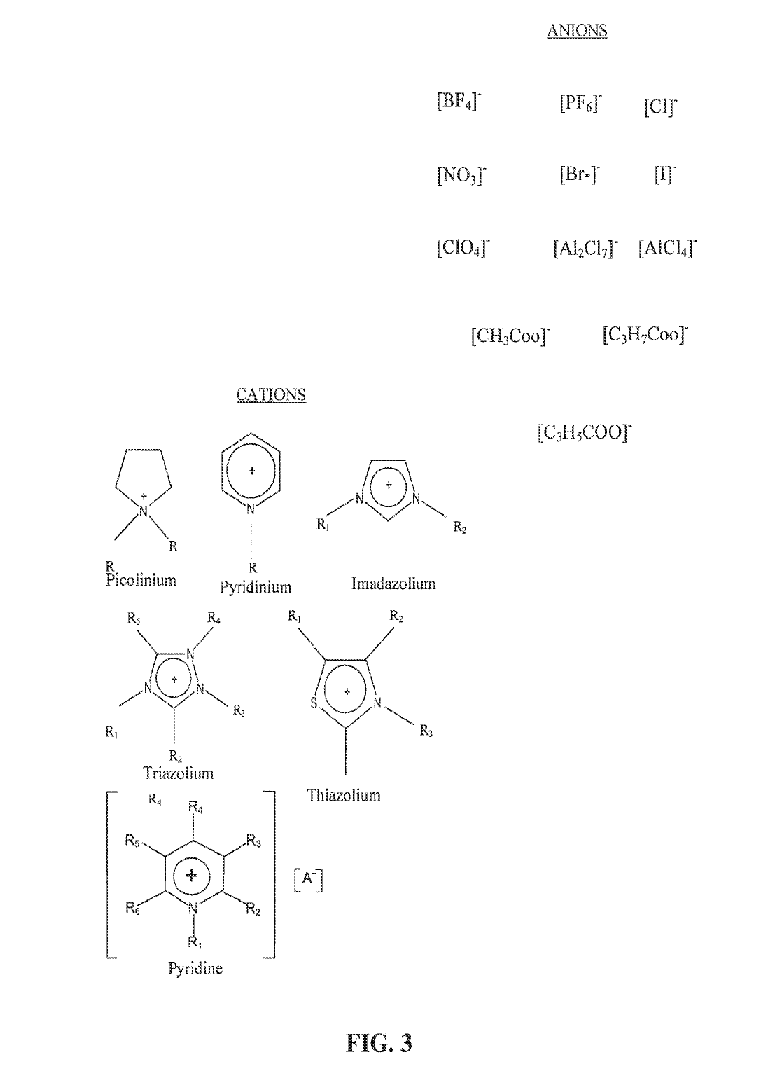

Any ionic liquid capable of disrupting the hydrogen bonding structure to reduce the crystallinity of cellulose in the biomass can be used in the treatment methods described herein may comprise a cation structure that includes imidazolium, pyrroldinium, pyridinium, phosphonium, ammonium, or a combination thereof and all functionalized analogs thereof. For example, the structure of triazolium as shown in FIG. 3 wherein each of R1, R2, R3, R4, and R5 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A may be a halide, hydroxide, formate, acetate, propionate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

The IL may be represented by the structure:

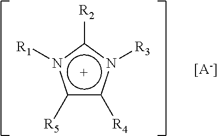

Each of R1, R2, R3, R4, and R5 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

The IL may be represented by the structure:

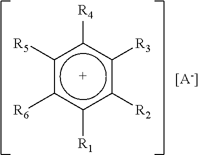

Each of R1, R2, R3, R4, R5, and R6 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate

Another example of the structure of IL is shown in FIG. 3 pyridine wherein each of R1, R2, R3, R4, R5, and R6 may be hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A may be a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate. The halide can be a chloride, fluoride, bromide or iodide.

Also an ionic liquid mixture with a composition described by Equation 1 may be used in the methods and systems described herein.

$${\sum }_{n = 1}^{20} {{[ {C}^{+} ]}_{n} [ {A}^{-} ]}_{n}$$

C⁺ denotes the cation of the IL and A⁻ denotes the anionic component of the IL In Equation 1. Each additional IL added to the mixture may have either the same cation as a previous component or the same anion as a previous component, of differ from the first only in the unique combination of the cation and anion. For example, consider below the five component mixture of ILs in which common cations and anions are used, but each individual IL component is different:

[BMIM⁺][Cl⁻]+[BMIM⁺][PF₆₋]+[EMIM⁺][Cl⁻]+[EM-IM⁺][PF₆₋]+[EMIM⁺][BF₄⁻]

The final mixture of ionic liquids will vary in the absolute composition as can be defined by the mole percent of various functionalized cations and anions. Therefore, the mixture may be comprised of varying weight percentages of each utilized component, as defined by Equation 1. The use of several such representative solvents for treating biomass may be 1-Ethyl-3-Methylimidazolium Propionate (EMIM-Pr) as described in U.S. Pat. No. 8,030,030. Also the ionic liquid 1-(4-sulfonic acid)butyl-3-methylimidazolium hydrogen sulfate may be used.

The ionic liquid for use in the methods described herein include but are not limited to 1-Butyl-3-methylimidazolium alkylbenzenesulfonate, 1-Ethyl-3-methylimidazolium alkylbenzenesulfonate, 1-Butyl-3-methylimidazolium acesulfamate, 1-Ethyl-3-methylimidazolium acesulfamate, 1-Ethylpyridinium chloride, 1-Butylimidazolium hydrogen sulfate, 1-Butyl-3-methylimidazolium hydrogen sulfate, 1-Butyl-3-methylimidazolium methyl sulfate, 1,3-Dimethylimidazolium methyl sulfate, 1-Butyl-3-methylimidazolium methanesulfonate, 1-Ethyl-3-methylimidazolium acetate, 1-Butyl-3-methylimidazolium acetate, 1-Octyl-3-methylimidazolium acetate, 1-(2-(2-Hydroxy-ethoxy)ethyl)-imidazolium acetate, 1-(2-(2-Methoxy-ethoxy)ethyl)-3-ethylimidazolium acetate, 1-(3,6,9,12-Tetraoxatridec-1-yl)-3-ethylimidazolium acetate, 1-(3,6,9,12,15,18,21-Heptaoxadocos-1-yl)-3-ethylimidazolium acetate, 1-(2-(2-Methoxy-ethoxy)ethyl)-triethylammonium acetate, (2-Hydroxy-ethyl)-dimethylammonium acetate, (2-Methoxyethyl)-dimethylammonium acetate, Tetramethylguanidinium acetate, Tetramethylguanidinium propionate, 1-Butyl-3-methylimidazolium formate, Tetrabutylphosphonium formate, Tetrabutylammonium formate, 1-Hexyl-3-methylimidazolium trifluoromethanesulfonate, 1-Butyl-3-methylimidazolium chloride, 1-Ethyl-3-methylimidazolium chloride, 1-Butyl-3-methylimidazolium bromide, 1-Allyl-3-methylimidazolium chloride, 1-Butyl-2,3-dimethylimidazolium tetrafluoroborate, 1-Butyl-3-methylimidazolium tetrafluoroborate, 1-Butyl-3-methylimidazolium hexafluorophosphate, 1-Butyl-1-methylpyrrolidinium hexafluorophosphate, 1-Ethyl-3-methylimidazolium diethyl phosphate, 1-Ethyl-3-methylimidazolium nitrate, 1,3-Dimethylimidazolium dimethyl phosphate, 1-Butyl-3-methylimidazolium dimethyl phosphate, 1-Methylimidazolium chloride, or combinations thereof.

The ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%. Also, the ionic liquid may be recovered and reused.

Pretreatment of Lignocellulosic Biomass with Ionic Liquids

The pretreatment of lignocellulosic biomass by using Ionic Liquids (ILs) facilitates efficient and rapid enzymatic hydrolysis of its carbohydrates. The goal of the IL pretreatment process is not achieving any dissolution of lignocellulose, but contacting it with the IL for sufficient time to redistribute lignin and swell the remaining biomass structure to enhance the hydrolysis rate and conversion of cellulose and hemicellulose to their constituent sugars. Following saccharification with an appropriate enzyme mix, capable of converting all the carbohydrates in the pre-treated biomass to sugars, most of the solids left behind in the saccharification reactor represent the lignin portion of the biomass. This provides a method of recovering the lignin from biomass. Also, ultra-filtration of the liquid portion of the hydrolysate, provides a means of recovering the hydrolysis enzymes for reuse from the sugar solution which is the precursor for the production of a number of fuels and chemicals.

Further, unlike most water-based pretreatment methods, IL-pretreatment does not produce sugars or their degradation products during incubation step because these are generally hydrolysis products that require presence of water. Sugars are produced only upon enzyme hydrolysis or acid hydrolysis. The absence of sugar degradation products that can prove inhibitory to the subsequent processing of the sugars (such as fermentation to alcohol and lactic acid) eliminates the need for the additional step of “conditioning”—in which these inhibitory products are removed—of the hydrolysate.

The time and temperature during the step of IL-incubation of the biomass is optimized to sufficiently swell matrices of the biomass to enhance the penetration of hydrolyzing enzymes and water during the hydrolysis step. The biomass may be incubated during the ionic liquid step until the cellulose and hemicellulose structure in the biomass is swollen at least 30% by volume compared to before the incubation step. The biomass may be incubated during the ionic liquid step until the cellulose and hemicellulose structure in the biomass is swollen at least 10%, 20, 30%, or 40% by volume compared to before the incubation step. The incubating step may comprise incubating the biomass for a time ranging from about 5 minutes to about 8 hours. The incubating step may be for a time ranging from about 1, 2, 3, 4, 5, 6, 7, or 8 hours. The incubating step may be for a time ranging from about 1-3, 4-6, or 6-8 hours. The incubating step may comprise incubating the biomass at a temperature ranging from about 50° C. to about 200° C. The incubating step may be at temperature ranging from about 50-100° C., 80-160° C., 50-125° C., 75-100° C., 100-150° C., 120-150° C., or 150-200° C. For example, the biomass may be incubated in the ionic liquid for about 30 minutes at 120° C. The biomass may be incubated in the ionic liquid for about 60 minutes at 120° C. The biomass may be incubated in the ionic liquid for about 10 minutes, 1 hour, or 3 hours at a temperature of about 120° C., 130° C., 140° C., or 140° C.

The liquid non-solvent for cellulose that is used for washing is water, an alcohol, acetonitrile or a solvent which dissolves the IL and thereby extracts the IL from the biomass. The alcohol may be ethanol, methanol, butanol, or propanol, and wherein the IL is recovered from the liquid non-solvent.

The method may further comprise reusing the recovered IL for treating more biomass.

The method may further comprise recovering the IL from the liquid non-solvent by a method selected from one or more of activated charcoal treatment, distillation, membrane separation, electro-chemical separation techniques, sold-phase extraction and liquid-liquid extraction.

### Recovery of IL/Dehydration of IL

The wash effluent may be collected and the ionic liquid dehydrated by the application of RF energy. The RF energy heats IL faster than it heats water because of a stronger dipole moment in IL. Without being bound to a specific theory, the inventors surprisingly discovered that the ions try to align with the electromagnetic (EM) (e.g., radiofrequency) waves, always changing a dipole moment. The IL heated by RF acts as a substrate for the water to heat and evaporate from the IL wash effluent. Thus, the wash effluent comprising a solvent and ionic liquid may be heated using RF energy. The RF energy drive off the water which may be collected and removed from the wash. The resultant ionic liquid is thus dehydrated (e.g., the water has been removed) and may be reused.

Thus, the ionic liquid is recovered from the liquid non-solvent by application of electromagnetic heating including but not limited to radiofrequency heating, that dehydrates the ionic liquid. The method may further comprise reusing the recovered IL for treating more biomass, optionally wherein at least 90, 91, 92, 93, 94, 95, 96, 97, 98, or 99% of the IL is recovered. After recovery, the ionic liquid may have a water content not exceeding about 1, 2, 3, 4, 5, 6, 7, 8, 9, 10, 11, 12, 13, 14, 15, 16, 17, 18, 19, 20, 21, 22, 23, 24, or 25%.

### Treatment of Biomass Using Combination of Ionic Liquids and Heating

lignocellulosic biomass may be treated using radio frequency heating in conjunction with ionic liquids (ILs) to facilitate efficient and rapid enzymatic hydrolysis of its carbohydrates. Exemplary ionic liquids (IL) and treatment methods are described in U.S. Pat. No. 8,030,030. lignocellulosic biomass may be treated utilizing heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, for effective and amenable conversion of biomass and derived products to renewable fuels, chemicals, and materials. For example, radio frequency heating provides uniform heating and penetration of the biomass.

A method for conversion of the carbohydrates of lignocellulose to sugars with improvements in yield and rate of sugar production using ionic liquid (IL) treatment in combination with RF heating. This treatment strategy substantially improves the efficiency (in terms of yield and reaction rates) of hydrolysis (e.g., saccharification) of lignocellulosic biomass. Other features of this IL-treatment method that have a major impact on the overall economics of sugar production from biomass, in contrast to prior art methods, are its (i) ability to process a variety of lignocellulosic biomass sources with ILs capable of disrupting native cellulose structure (ii) ability to handle large biomass to IL ratios during incubation (iii) ability to accomplish saccharification at very low enzyme loadings (iv) ability to perform well with large biomass particles (v) potential for total recovery (through facile means) and multiple reuse of the IL employed to treat the biomass, (vi) ability to produce a hydrolysate free of compounds that can inhibit the down-stream processing of the constituent sugars, (e.g., ethanol and lactic acid production), and (vii) allows for recovering most of the lignin in biomass following saccharification.

The biomass may be comminuted to smaller sized particles prior to mixing with an ionic liquid and treatment. The biomass may be fed into a chipper, grinder, chopper, shredder, or similar machine to be reduced in size. For example, the biomass may be ground, chopped, or otherwise comminuted to small particles about 0.1-2 mm.

The biomass may be comminuted to smaller sized particles. The biomass may be comminuted to smaller sized particles prior to mixing with an ionic liquid. The biomass may be comminuted to small particles about 0.1-20 mm, 0.1-2 mm, or about 5 mm in size.

The biomass may be processed at a high level of loading. The biomass may comprise high solids loadings at least about 10%, 20%, 30%, 40%, 50%, 60%, 70%, 80%, 90%, or 95% w/w. The biomass may be loaded at high solids loading at approximately 30% w/w.

For example, a method for producing sugars from biomass, including but not limited to wheat straw, waste rice straw, kudzu, agricultural waste, municipal waste, corn stover, wood waste, agricultural residues including but not limited to corn stover, wheat straw, bagasse, rice hulls, or rice straw; wood and forest residues including but not limited to pine, poplar, Douglas fir, oak, saw dust, wheat straw, paper/pulp waste, or wood fiber; herbaceous energy crops including but not limited to switchgrass, reed canary grass, or miscanthus; lingocellulosic biomass comprising lignin, cellulose, and hemicellulose; and plant biomass. The biomass may be added to a high solids loading (e.g., >30% w/w). The biomass is mixed with ionic liquid (IL) to swell the biomass but not dissolve it in the IL and heated using radio frequency (RF) energy. Both the mixing with ionic liquid and heating with RF may be monitored for sufficient penetration and uniform heating and the conditions (e.g., time, pressure, heat, intensity of RF energy) may be adjusted as necessary to maintain sufficient penetration and uniform heating of the biomass. Optionally, after the application of RF heating, ultrasonics, electromagnetic heating (EM) (e.g., radiofrequency), convective, conductive heating, or combinations thereof may be used to maintain the temperature of the biomass.

### Electromagnetic (EM) Wave Heating

Biomass products at high solids loadings are relatively poor thermal conductors and most conventional thermal treatment processes rely on heat penetration by conduction from the outside to the inside of the product (surface heating). The processing times can be unacceptably long in industrial scale processing operations. Dielectric heating by microwave or radio-frequency (RF) energy shortens thermal processes because heat is generated by direct interaction between electromagnetic energy and the products. RF-heating, in a similar manner to microwave heating, generates heat volumetrically throughout the product. However, RF treating differs from microwave treatment in that the product is placed between two parallel electrodes and an RF field may be generated in a directional fashion at right angles to the surface of the electrodes.

In addition, the mechanism of dielectric heating with RF field is different from microwave (MW) heating. MW heating occurs mainly via frictional heat generated from the dipolar rotation of free water molecules whereas the predominant mechanism of heating RF is via the depolarization of solvated ions. MW and RF heating also differ in a number of other respects. As frequency and wavelength are inversely proportional, RF (lower frequency) wavelengths (i.e., 11 m at 27.12 MHz in free space) are much longer than MW (higher frequency) wavelengths (i.e., 0.12 m at 2450 MHz in free space). As electrical waves penetrate into materials attenuation occurs, with the result that the energy of the propagating wave decreases exponentially. Penetration depth (dp) is defined as the depth into the material to which the energy is reduced to 1/e (1/2.72) of the surface energy value. This dp is proportional to wavelength. The free-space wavelength in the RF range (e.g., 13.56, 27.12 and 40.68 MHz) is 20-360 times longer than that of commonly used microwave frequencies (e.g., 915 and 2450 MHz), allowing RF energy to penetrate products more deeply than microwave energy. During RF heating, electromagnetic power can penetrate much deeper into samples without surface over heating or hot/cold spots developing which are more likely to occur with MW heating. Thermal processing with RF heating is, therefore, suitable for processing large products/processes. Wang, et al. (2003) Journal of Food Science 68(2): 539-544.

RF heating offers advantages of more uniform heating over the sample geometry due to both deeper level of power penetration and also simpler more uniform field patterns compared to MW heating. In contrast to RF-heating, higher frequency microwaves may provide for greater heating intensity, however, have limits for biomass products when they cannot penetrate deeply enough or provide uniform heating. Power penetration depth decreases with shorter wavelength that is, increasing frequencies. Penetration depths at radio frequencies are of the order of meters and, unless the loss factor is extremely high, through heating may be assured. In the microwave region, on the other hand, the penetration depths become very small, especially when a material is very wet. The wavelength at the RF heating frequencies designated by the Federal Communication Commission (FCC) for industrial heating is 22 to 360 times as great as that of the 2 commonly used microwave frequencies, which allows RF energy to penetrate dielectric materials more deeply than microwaves. Thus, radio frequency heating shows unexpected results in biomass treatment and dielectric materials processing at larger scales and higher levels of solids loading (e.g., about >20% w/w and about >70% w/w).

RF heating has been proven to allow rapid heat transfer throughout dielectric materials as the volumetric heating does not depend on heat transfer through the surface and continues through the boiling point of water and beyond. RF heating is a heating technology that allows for rapid, uniform heating throughout a medium. This technology generates greater energy within the product and throughout its mass simultaneously due to frictional interactions of polar dielectric molecules rotating to an applied external electric field. RF dielectric heating offers several advantages over conventional heating methods in food application, including saving energy by increasing heat efficiency, achieving rapid and even heating, reducing checking, avoiding pollution as there are no byproducts of combustion. Cathcart and Park (1946) first studied the use of RF heating to thaw frozen eggs, fruits, vegetables, and fish. Radio frequency dielectric heating is now widely used in industrial applications such as drying wood logs, textile products (e.g., spools, rovings, skeins), final drying of paper, final dehydration of biscuits at outlets of baking ovens, and melting honey (Barker 1983; Orfeuil 1987).

The problem however with a straight forward use of electromagnetic (EM) (e.g., radiofrequency) wave heating of biomass and ionic liquid complex is the generation of runaway temperatures. In typical uses described above, water absorbs the impinging energy and helps raise the temperature of the complex. As water evaporates, the electromagnetic (EM) (e.g., radiofrequency) waves pass through that part of the material without further energy dissipation. With ionic liquids or complexes containing ions, that do not evaporate or are not meant to evaporate, the setup needs to be much more specifically controlled. The control may include several sensors (e.g., thermocouples, nano-sensors, flow sensors, or other types of sensors) that relay the local conditions so the electromagnetic (EM) (e.g., radiofrequency) unit for that region can be appropriately controlled (e.g., turned on/off or set to a different frequency/power). This setup as such can be used in treatment, hydrolysis (e.g., acid hydrolysis or enzymatic hydrolysis or IL based or a combination thereof) or other reaction environments, whenever the loading of biomass with respect to the other components in the complex is relatively high.

The heating for the treatment of the biomass may comprise two phases: (1) Initial Phase where RF energy is applied to rapidly heat the biomass and (2) Maintenance Phase where of ultrasonics, electromagnetic irradiation (EM) (e.g., radiofrequency), convective, conductive heating, or combinations thereof is applied to maintain the heat of the biomass.

The heating of the biomass by RF may be monitored by a microcontroller and maintained within set parameters of temperature and pressure. For example, the biomass may be maintained at a pre-determined temperature, and additional RF applied when the temperature of the biomass falls below this target temperate and RF is discontinued when the temperature of the biomass exceeds the target temperature. This process may be repeated to maintain an average temperature of the biomass during RF heating.

The inventors surprisingly found that the RF heating may rapidly, uniformly, and effectively heat the biomass allowing for a faster processing time of the biomass. Also, the use of RF heating unexpectedly allowed for higher solids loading (e.g., >30% w/w).

Radio frequency (RF) may comprise a frequency between at least about 3-30 Hz, 30-300 Hz, 300-3000 Hz, 3-30 kHz, 30-300 kHz, 300 kHz-3 MHz, 3-30 MHz, or 30-300 MHz. The radio frequency (RF) may be about 13, 13.56, 27, 27.12, 40, or 40.68 MHz.

The biomass may heated to a temperature of at least about 10° C., 20° C., 30° C., 40° C., 50° C., 60° C., 70° C., 80° C., 90° C., 100° C., 120° C., 130° C., 140° C., 150° C., 200° C., 300° C., 400° C., 60° C.-130° C., 80° C.-175° C., 130° C.-150° C., or 100° C.-240° C.

The radiofrequency may penetrate RF penetrates the biomass to about 0.001 to 2.0 meters thickness. The radiofrequency heating may occur with agitation, either intermittent or continuous.

The biomass may be heated with RF for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The biomass may be heated with RF for at least about 1-60 seconds, 1-30 seconds, 1-20 seconds, 5-10 seconds, or 1-10 seconds. The biomass may be heated with RF for at least about 10, 20, 30, 40, 50, 60 seconds. The biomass may be heated with RF for at least about 1, 2, 3, 4, 5, 6, 7, 8, 9, 10, 20, 30, 40, 50, 60, 70, 80, 90, 100, 110, 120, 130, 140, 150, 160, 170, or 180 minutes. The biomass may be heated with RF for at least about 1, 2, 3, 4, 5, 6, 7, 8, 9, 10, 11, 12, 13, 14, 15, 16, 17, 18, 19, 20, 21, 22, 23, or 24 hours. The biomass may be heated with RF for at least about 1, 2, 3, 4, 5, 6, 7, 8, 9, or 10 days. The biomass may be heated with RF for at least about 5-10 seconds, 10-30 seconds, 10-30 minutes, 1-30 minutes, 5-30 minutes, 1-20 minutes, 20 minutes to 2 hours, 5 minutes to 3 hours, 5 minutes to 2 hours, 1-4 hours, 2-4 hours, 1-2 hours, or 3-4 hours.

The biomass may treated at a pressure of at least about 1, 2, 3, 4, 5, 6, 7, 8, 9, 10, 11, 12, 13, 14, 15, 16, 17, 18, 19, 20, 30, 40, 50, 60, 70, 80, 90, or 100 atmospheres (atm).

The ultrasonics used in the methods described herein may be sound waves with high frequency about between 15-40 kHz, 20-30 kHz, 25-35 kHz, or about 15, 20, 30, 35, 35, or 40 kHz) with an amplitude between about amplitude about between 0.0001-0.025 mm. The ultrasonics heating may occur with agitation, either intermittent or continuous.

The biomass may be heated at a power of 100-1,000 W, 1 KW-10 KW, or 5 KW-1 MW.

The biomass may be comminuted to smaller sized particles. The biomass may be comminuted to smaller sized particles prior to mixing with an ionic liquid. The biomass may be comminuted to small particles about 0.1-20 mm, 0.1-2 mm, or about 5 mm in size.

A method for disruption of the structure of a lignocellulosic biomass comprising lignin, cellulose and hemicellulose and treating the disrupted biomass, may comprise incubating the biomass in an ionic liquid (IL) for a sufficient time and temperature to swell the cellulose and hemicellulose without dissolution of the biomass in the IL, optionally heating by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof; washing the swelled IL-incubated lignocellulosic biomass with a liquid non-solvent for cellulose that is miscible with water and the IL; and treating the incubated and washed lignocellulosic biomass with chemical or biochemical reagents to effect the conversion of the swollen biomass to useful chemicals.

The biomass may heated by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof to a temperature of at least about 10° C., 20° C., 30° C., 40° C., 50° C., 60° C., 70° C., 80° C., 90° C., 100° C., 120° C., 130° C., 140° C., 150° C., 200° C., 300° C., 400° C., 60° C.-130° C., 80° C.-175° C., 130° C.-150° C., or 100° C.-240° C.

The biomass may be heated by radio frequency, ultrasonics (e.g., sound waves with high frequency about between 15 kHz to 40 kHz, or 20 kHz and low amplitude about between 0.0001-0.025 mm), electromagnetic irradiation (EM) (e.g., radiofrequency), convective, conductive heating, or combinations thereof, for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 seconds. The biomass may be heated for at least about 1-60 seconds, 1-30 seconds, 1-20 seconds, 5-10 seconds, or 1-10 seconds. The biomass may be heated for at least about 10, 20, 30, 40, 50, 60 seconds. The biomass may be heated for at least about 1, 2, 3, 4, 5, 6, 7, 8, 9, 10, 20, 30, 40, 50, 60, 70, 80, 90, 100, 110, 120, 130, 140, 150, 160, 170, or 180 minutes. The biomass may be heated with RF for at least about 1, 2, 3, 4, 5, 6, 7, 8, 9, 10, 11, 12, 13, 14, 15, 16, 17, 18, 19, 20, 21, 22, 23, or 24 hours. The biomass may be heated for at least about 1, 2, 3, 4, 5, 6, 7, 8, 9, or 10 days. The biomass may be heated for at least about 5-10 seconds, 10-30 seconds, 10-30 minutes, 1-30 minutes, 5-30 minutes, 1-20 minutes, 20 minutes to 2 hours, 5 minutes to 3 hours, 5 minutes to 2 hours, 1-4 hours, 2-4 hours, 1-2 hours, or 3-4 hours.

The biomass may be processed at a high level of biomass. The biomass may comprise high solids loadings at least about 10%, 20%, 30%, 40%, 50%, 60%, 70%, 80%, 90%, or 95% w/w. The biomass may be loaded at high solids loading at approximately 30% w/w. The inventor surprisingly discovered that the use of electromagnetic heating (e.g., radiofrequency heating, variable frequency electromagnetic heating) allows for the treatment of biomass at high solids loading levels, e.g., >30% w/w.

The electromagnetic heating used in the methods and systems described herein may be a variable frequency in the electromagnetic spectrum (e.g., radiofrequency).

For example, a method for conversion of the carbohydrates of lignocellulosic biomass to sugars may comprise mixing lignocellulosic biomass in an ionic liquid (IL) to swell but not dissolve the biomass; applying ultrasonics, electromagnetic (EM), preferably radio frequency (RF), convective, conductive heating, or combinations thereof, to the lignocellulosic biomass to heat the lignocellulosic biomass at a temperature range of about 50-220° C.; washing the treated lignocellulosic biomass; subjecting said lignocellulosic biomass to mild alkaline treatment to release lignin from the cellulosic components; washing the treated lignocellulosic biomass; recovering the lignin, cellulose, and hemicellulose; separating the lignin, cellulose, and hemicellulose; hydrolyzing the cellulose to yield sugars; and hydrolyzing the hemicellulose to yield sugars.

### Mild Alkaline Treatment

Following treatment (e.g., incubation with ionic liquid and heating), the treated biomass may be washed to remove the ionic liquid and then treated with alkaline treatment including but not limited to soda, Kraft, sulphite, and/or alkaline process (e.g., NaOH, ammonia, LiOH, Mg(OH)₂, Al(OH)₃, Ca(OH)₂, H₂O₂, NaS, NasCo₃) under extremely mild conditions.

Soda pulping was one of the first chemical pulping methods invented in 1851 by Burgess (USA) and Watts (England). Though Kraft or sulfite pulping process took over soda pulping process for generating high strength pulp, soda pulp offers advantages for environmental concerns. As sodium hydroxide is the only used chemical in soda pulping, air pollutants such as hydrogen sulfide (from Kraft process) and sulfur dioxide (from sulfite process) are not observed. Process conditions range from treating biomass with 11 to 22% NaOH at 160-200° C., 70 to 130 Psi for 4 to 5 hours of treatment.

Alkaline pretreatments of biomass have been attempted using reagents ammonium, sodium hydroxide and calcium hydroxide starting as early as 1972 to improve the digestibility of cellulose/biomass as efficient feed stock for livestock with improvements of up to 13 to 90%. NaOH, ammonia and Ca(OH)₂ yielded similar digestibility yields based on the studies.

Numerous alkaline pretreatment techniques have been developed for biomass pretreatment over the years. These include sodium hydroxide, sodium carbonate, calcium hydroxide, aqueous ammonia. Alkaline pretreatments delignify biomass and solubilize hemicellulose. The effectiveness of alkaline pretreatment depends on the extent of lignin present. The mechanism of alkaline hydrolysis is believed to be the saponification of intermolecular ester bonds cross-linking xylan hemicelluloses and other components such as lignin. The porosity of the lignocellulosic biomass increases with the removal of the crosslinks thereby increasing the digestibility of cellulose. For example, U.S. Pat. No. 5,693,296 describes the use of several pretreatment conditions.

The present invention provides sequential pretreatments of ionic liquids followed by mild alkaline treatment (e.g., using in either single or combination of NaOH, ammonia, LiOH, MgOH, Ca(OH)₂, H₂O₂, NaS, Na₂CO₃). Ionic liquid pretreatment swells the biomass and whereas mild alkaline treatment conditions separates cellulose pulp from lignin. The decrystallized swelled cellulosic and segregated lignin fractions thus obtained can be rapidly converted to valuable fuels and chemicals in a catalytic process. The inventors surprisingly discovered that the mild alkaline treatment was sufficient to release the lignin from the cellulose pulp and allows greater access to the cellulose pulp by enzymes during the later hydrolysis step. This lead to unexpected improvement in the recovery of enzymes, the reduction of hydrolysis time, and greater yields of sugars. Further the lignin may be recovered. The recovered lignin may be subject to further processing to produce chemicals because it may not be signifigantly degraded or oxidized in contrast to prior methods.

The alkaline agents may be used at a concentration of about 10-20% by weight. The alkaline agents may be used at a concentration of about 1, 2, 3, 4, 5, 6, 7, 8, 9, 10, 11, 12, 13, 14, 15, 16, 17, 18, 19, or 20% by weight. The alkaline agents may be used at a concentration of about 2, 3, 4, or 5% by weight. The alkaline agent may be added at about 0.1, 0.2, 0.25, 0.3, 0.4, 0.5, 0.6, 0.7, 0.8, 0.9, 1, 2, 2.1, 2.2, 2.3, 2.4, 2.5, 3, 4, 5, 6, 7, 8, 8.25, 8.5, 8.75, 9, 10, 11, 12, 13, 14, 15, 16, 16.2, 16.4, 16.5, 16.65, 16.7, 16.8, 17, 18, 19, or 20% by weight. The alkaline agent may be added at about 0.1-10%, 0.1-0.5%, 0.1-5%, 2-8%, 5-15%, 10-20% by weight.

The mild alkaline condition may comprise about a pH 8-13, pH 8-10, pH 9-11, pH 9-10, pH 10-11, pH 9.5-10.5. The pH may be about 8, 8.25, 8.5, 8.75, 9, 9.25, 9.5, 9.75, 10, 10.25, 10.5, 10.75, 11, 11.5, 12, 12.5, or 13.

The alkaline treatment may occur at a temperature of at least about 10° C., 20° C., 30° C., 40° C., 50° C., 60° C., 70° C., 40° C.-60° C., 50° C.-70° C., 50° C.-60° C., or 40° C.-70° C. The alkaline treatment may occur at a temperature of at least about 40° C., 50° C., 70° C., 40° C.-60° C., 50° C.-70° C., 50° C.-60° C., or 40° C.-70° C. The temperature may be at least about 10° C., 20° C., 30° C., 40° C., 50° C., 60° C., 70° C., 73° C., 75° C., 78° C., or 80° C. The temperature may be at least about 10-50° C., 30-70° C., 40° C.-60° C., 50° C.-70° C., 50° C.-80° C., 40° C.-80° C., 50-80° C., 50° C.-70° C., 50° C.-60° C., or 40° C.-70° C.

The biomass may undergo alkaline treatment for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 minutes. The biomass may undergo alkaline treatment for at least about 1-60, 1-70, 1-75, 1-80, 1-30, 1-20, 5-10, or 1-10 minutes. The biomass may undergo alkaline treatment for at least about 10, 20, 30, 40, 50, or 60 minutes. The biomass may undergo alkaline treatment for at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 minutes. The biomass may undergo alkaline treatment for at least about 5-10, 10-30, 10-30, 1-30, 5-30, 1-20, or 30 minutes. The biomass may undergo mild alkaline treatment for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 minutes. The biomass may undergo mild alkaline treatment for about 1-60, 1-30, 1-20, 5-10, or 1-15 minutes.

These conditions are less harsh than the prior methods of soda/kraft pulping conditions which generally last for hours (e.g., 3-5 hours) at high temperature (e.g., 120-150° C.) for weeks (e.g., 3-4 weeks). The methods described herein also allow for the separation and recovery of the cellulose, hemicellulose, and lignin in contrast to prior methods. In prior methods, the lignin and hemicellulose was largely lost or degraded. Further, the pretreatment methods described herein do not require an oxidation step to prepare the biomass for hydrolysis.

### Conversion to Value Added Products

The treated biomass may be washed and then undergo cellulose hydrolysis (cellulolysis) to break down the cellulose and hemicellulose into sugars and free lignin and/or proteins. In the hydrolysis process, the cellulose and hemicellulose may undergo a chemical treatment (e.g., using acids—acid hydrolysis) or a biochemical treatment (e.g., enzyme hydrolysis). The sugars may then be separated from residual materials (e.g., lignin, proteins). The lignin may be recovered in a condition that allows for its conversion to chemicals. The sugar solution may then be converted to chemicals (e.g., ethanol, lactic acid, succinic acid). Treatment with has a major influence on the reducing the cost in both prior (e.g., size reduction) and subsequent (e.g. enzymatic hydrolysis) operations in sugar production and improving yields. The sugars yielded by the hydrolysis, acid hydrolysis and/or enzyme hydrolysis, include but are not limited to glucan, xylan, arabinan, mannan, alactan, glucose, sucrose, hexose, and combinations thereof. The sugars yielded by the hydrolysis, acid hydrolysis and/or enzyme hydrolysis, include but are not limited to glucan, xylan, arabinan, mannan, alactan, glucose, sucrose, hexose, and combinations thereof, may undergo further processing by acid hydrolysis to yield chemicals including but not limited to succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

The following processes may be used to convert biomass (e.g., cellulose, hemicellulose) to value added chemicals (e.g., ethanol). See Corma, et al. (2007) “Chemical Routes for the Transformation of Biomass into Chemicals.” Chem. Rev. 107: 2411-2502. The methods described herein separates the biomass into its main constituents: cellulose, hemicellulose, and lignin. The cellulose and hemicellulose may then be converted (e.g., acid hydrolysis and/or enzyme hydrolysis) to sugars. For example, the hemicellulose may be converted to five and six carbon sugars (e.g., xylose, arabinose) and the cellulose may be converted to six-carbon sugars (e.g., glucose.) The sugars may then be fermented to product products (e.g., ethanol). The proteins and/or lignin may be converted to energy, fuel, plastics, or binders. The cellulose and hemicellulose may undergo a hydrolysis process (cellulolysis), either chemical treatment (e.g., acids) or a biochemical treatment (e.g., enzymatic digestion). Some methods for the chemical processing of cellulose, lignocellulose, and other biomass into chemicals are known in the art. See, e.g., Kobayashi, et al. (2012) Catal. Sci. Technol. 2: 869-883; Ishikawa & Saka (2001) “Chemical Conversion of Cellulose as treated in supercritical methanol.” Cellulose 8(3): 189-195; Tao, et al. (2011) “Catalytic Conversion of cellulose to chemicals in ionic liquid.” Carbohydrate Research 346(1): 58-63; Tao, et al. (2011) Carbohydrate Research 346(1): 58-63; and Binder & Raines (2009) J. Am. Chem. Soc. 131: 1979-1985. These methods may be used in conjunction with the treatment and treatment methods described herein.

### Chemical Conversion to Value Added Products

The chemical treatment may comprise incubation with acids under heat and pressure or a concentrated acid hydrolysis process (e.g., Scholler process). See also Robinson (1995) “A Mild, Chemical Conversion of Cellulose to Hexane and Other Liquid Hydrocarbon Fuels and Additives,” ACS Fuel Chemistry Preprints 40(3): 729 and Binder & Raines (2010) PNAS 107(10): 4516-4521. The cellulose may be treated with alkaline peroxide and then treated with enzymes to break down the cell wall. For example, the biomass may be treated with an ionic liquid to convert the sugars (e.g., glucose and fructose) into 5-hydroxymethylfurfural (HMF). Oxidation of HMF yields 2,5-furandicarboxylic acid.

In other processes, the cellulose and hemicellulose may be converted to 5-hydroxymethylfurfural (HMF) that may be used as a raw material for plastics and fuels. A metal chloride (e.g., chromium chloride) may be used with an ionic liquid to convert the sugars (e.g., glucose and fructose) into HMF. The chemical, a metal chloride known as chromium chloride, converted sugar into highly pure HMF. The metal chlorides and ionic liquid may be reused. Oxidation of HMF yields 2,5-furandicarboxylic acid, which may be used as a replacement for terephthalic acid in the production of polyesters (e.g., polyethylene terephthalate (PET)). See Zhao, et al. (2007) Science 316(5831): 1597-1600.

Further, the cellulose may be degraded by the use of cooperative ionic liquid pairs for combined dissolution and catalytic degradation of cellulose into 2-(diethoxymethyl)furan. See Long, et al. (2011) Green Chem. 13: 2334-2338.

Catalysts may be used in the methods described herein to increase the reaction rate of the reactions. For example, alkali and alkaline earth metal chlorides, and transition metal chlorides (e.g., CrCl₃, FeCl₂, and CuCl₂), and IIIA metal chlorides (e.g., AlCl₃) may be used as catalysts. See, e.g., Peng, et al. (2010) Molecules 15: 5258-5272. Additionally, CoSO₄ may be used as a catalyst in conjunction with an ionic liquid.

Additionally, the sugars produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

Also, the treated biomass produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. Also, the chemical processing of the treated biomass may yield gas productions including but not limited to methane, ethane, CO, CO₂, and H₂.

Also, the treated biomass produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. Also, the biochemical processing of the treated biomass may yield gas productions including but not limited to methane, ethane, CO, CO₂, and H₂.

Further, the hemicellulose may be converted to xylose and then to ethanol, xylitol, plastics. The lignin may be converted to fuel, plastics, and binders. The cellulose may be converted to glucose and pulps.

### Biochemical Conversion to Value Added Products (Enzyme Hydrolysis)

The biochemical reagent used to convert the washed biomass is an added enzyme. The treating step may comprise adding a buffer comprising enzymes capable of hydrolyzing both cellulose and hemicellulose to the incubated and washed biomass to hydrolyze the cellulose and hemicellulose to sugar. The aqueous buffer may comprise enzymes may comprise an enzyme mixture of cellulases, endo-glucanases, exo-glucanases, and 1-beta-glucosidases. The method may further comprising recovering the enzymes from the hydrolyzed biomass. The enzyme mixture may further may comprise xylanases or arabinases.

In enzymatic hydrolysis, the cellulose is digested into sugar molecules by cellulase enzymes. The lignocellulosic materials may be enzymatically hydrolyzed at mild conditions (e.g., 50° C. and pH 5) to breakdown the cellulose. The methods described herein combine ionic liquid pretreatment with mild alkaline treatment to prepare lignocellulosic biomass for enzymatic hydrolysis to produce monomeric sugars and then chemicals, fuels, and materials. The combination of the ionic liquid pretreatment with mild alkaline treatment has the unexpected result of releasing the lignin from the cellulose and hemicellulose which, in turn, allows for less enzyme to be used for a shorter period of time with greater recovery of the enzyme. Without being bound to a particular theory, the inventors suggest that the release and removal of the lignin from the lignocellulosic biomass allows for greater access of the enzymes to hydrolyze the cellulose and hemicellulose and also eliminates excess lignin which may bind and thus, interfere with, the enzymes.

The enzymes may be used at an amount of 0.5, 1, 2, 3, 4, or 5% enzymes. The enzymes may be used at an amount of 0.5%, 1%, or 2%.

The hydrolysis by enzymes may be for about 1, 2, 3, 4, 5, 6, 7, 8, 9, 10, 11, or 12 hours. The enzymatic hydrolysis may be for about 8-12 hours. The enzymatic hydrolysis may be for about 1-2, 2-4, 6-10, or 6-12 hours. This time is greatly reduced from the 24-36 hours or 72-96 hours hydrolysis times of prior hydrolysis methods.

The enzymes may be recovered after hydrolysis. For example, about 60, 70, 80, 90, 91, 92, 93, 94, 95, 96, 97, 98, 99, or 100% of the enzymes may be recovered and, optionally, reused. About 80% or 90% of the enzymes may be recovered and, optionally reused. This is in contrast to prior methods were less than 50% of the enzymes may be recovered. For example, in prior methods after 36 hours of enzyme hydrolysis about 50% of the enzyme was recovered. In contrast, the methods described herein allows for 8-12 hours of enzyme hydrolysis with over 90% recovery of the enzyme. Thus, the methods described herein allow for the run of several “cycles” of enzyme hydrolysis per day thus allowing for an unexpectedly high yield of monomeric sugars from treated biomass. For example, in prior methods over the course of 5 days, 3-4 cycles may have been run with about 50% recovery. In contrast, the methods described herein, over the course of 5 days allows for over 20 cycles with 80-90% recovery of enzymes. This allows for an unexpectedly greater yield of sugars, with less enzymes over the same period of time, resulting in cost savings.

Further, alkali pretreatments at high temperatures and a few hours of processing result in hemi-cellulose and lignin predominantly staying together and cellulose obtained in a separate stream. Alkali pretreatments at milder conditions 50-70° C. needs days to weeks for pretreatments and result in separate lignin stream and cellulose, hemi-cellulose stream. In contrast, the methods described herein comprise mild conditions and short duration processing times with the production of a lignin rich stream and a separate cellulose, hemi-cellulose rich stream. Thus, the methods described herein involve short duration (e.g., minutes) and retain hemicellulose with cellulose stream.

In the enzymatic treatment of the treated biomass, the biomass may be heated to at least about 50-100° C. In the enzymatic hydrolysis of the cellulose and hemicellulose, the biomass may be heated to about 51° C., 52° C., 53° C., 54° C., 55° C., 56° C., 57° C., 58° C., 59° C., 60° C. 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

For example, cellobiohydrolase, exo-1,4-β-glucanase, do-beta-1,4-glucanase, beta-glycosidase, endocellulase, exocellulase, cellobiase, and beta-1,4-glucanase may be used for enzymatic digestion of cellulose. The hemicellulases include but are not limited to laminarinase, lichenase, β-xylosidase, xylanases (e.g., endo-1,4-β-xylanase, xylan 1,4-β-xylosidase, xylan endo-1,3-β-xylosidase, xylan 1,3-β-xylosidase), α-L-arabinofuranosidase, arabianan endo-1,5-α-L-arabinosidase, mannananses (e.g., mannan endo-1,4-β-mannosidase, mannan 1,4-β-mannosidase, mannan 1,4-β-mannobisosidase, mannan endo-1,6-β-mannosidase), galactanases, and xylanase may be used for enzymatic digestion of hemicellulase. Jeffries “8. Biodegradation of lignin and hemicelluloses.” Biochemistry of Microbial Degradation pages 233-277. The cellulase, xylanase, and hemicellulase enzymes may be recombinant, including those expressed by recombinant fungi. See Lynd (1996) Annu Rev Energy Environ 21: 403-465.

In the combined hydrolysis and fermentation approach, the cellulose and hemicellulose may be incubated with Clostridium thermocellum which uses its a complex cellulosome to break down cellulose into ethanol, acetate, and lactate.

For ethanol production, the cellulose may undergo cellulolysis processes or gasification. In cellulolysis, the treated lignocellulosic biomass undergoes hydrolysis and then the cellulose may be treated by microbial fermentation. For example, the cellulose may be incubated with Saccharomyces cerevisiae, Zymomonas mobilis, and Escherichia coli, including recombinant microbes, to ferment xylose and arabinose to produce sugars and ethanol. See Jeffries & Jin (2004) Appl Microbiol Biotechnol 63(5): 495-509. The gasification process, a thermochemical approach, the cellulose and hemicellulose is converted into synthesis gas. The carbon monoxide, carbon dioxide and hydrogen may then be incubated with Clostridium ljungdahlii. The Clostridium ljungdahlii ingests carbon monoxide, carbon dioxide, and hydrogen to produce ethanol and water.

Thermostable enzymes may be used in the hydrolysis step. Thermostable enzymes may be stable and active up to about 70° C., as opposed to 55° C. for most commercially available enzymes.

### Chemical Conversion to Chemicals and Fuels (Acid Hydrolysis)

Hydrolysis of biomass using dilute acids or concentrated acids can rapidly hydrolyze biomass to sugars. However, the highly recalcitrance nature of biomass requires acids high concentrations of about 60-70% acids in order to produce cellulose sugars. About 60-90% of cellulosic sugars can be produced from concentrated acid hydrolysis above 70% concentrated acid hydrolysis. Sugars produced in this process are quite rapid. However, commercial implementation of this process in not feasible due to difficulty of procuring and operating highly acid resistant equipment, difficulty in recovering, concentrating, and reusing the acid.

Dilute acid (single stage or two stage hydrolysis) pretreatment of biomass substrates generally involve the use of 0.1-15% acids to hydrolyze cellulosic material at temperatures ranging from 300-600° C. and pressures up to 800 Psi. Most of these technologies employ a dilute acid hydrolysis under moderate temperature conditions (140-160° C.) to release the pentoses. In the second stage the temperatures are raised to 200-240° C. to facilitate the hydrolysis of cellulose and recovery of six carbon sugars. However, none of the pretreatments existed thus far, have been able to liberate C₆ sugars in higher yields effectively from lignocellulosic components under mild conditions. Nguyen, Q., Milestone Completion Report: Evaluation of a Two-Stage Dilute Sulfuric Acid Hydrolysis Process, Internal Report, National Renewable Energy Laboratory, Golden, Colo. 1998. At moderate operating conditions poor yields of sugars are obtained and at severe operating conditions such as above 200-240° C., major degradation products including humins and char are formed. Therefore, dilute acid processes have not been successful in liberating C₆ sugars from cellulosic materials in high yields.

Aqueous phase catalysis of regular lignocellulosic biomass (without IL conditioning) to generate products in high yields was not possible at moderate conditions (at severe conditions degradation and char forming takes place). In contrast, the processes described herein allow for rapid catalytic conversion of biomass to high value monomeric products in aqueous phase catalysis under moderate reaction conditions.

This invention provides an efficient biomass disruption/fractionation strategy employing sequential preparation, conditioning, and acid hydrolysis (a) can be used for treating any lignocellulosic biomass substrates, (b) results in efficient cellulosic material and lignin fraction generation at mild conditions (of temperature, pressure, time, chemical, solvents) enabling catalytic conversions of all constituents of biomass in a single or sequential steps (c) results in a de-crystalized and swollen cellulose for catalytic conversion to monomeric sugars or chemicals, (d) results in enhanced production of monomeric sugars, (e) results in a catalytically convertible lignin fraction for generation of aromatic chemicals, (f) results in purer fractions of cellulosic pulp and lignin fractions, (g) results in lower reagent, chemical and catalyst requirements, and (h) enables economic recovery of catalysts and chemicals.

Following treatment (e.g., incubation with ionic liquid and heating), the treated biomass may be washed to remove the ionic liquid and then treated with mild acidic treatment including but not limited to sulfuric acid, hydrochloric acid, phosphoric acid, nitric acid, maleic acid or solid acids under extremely acidic conditions.

The methods described herein to provide for dilute acid hydrolysis of lignocellulosic biomass to produce cellulosic sugars. The methods described herein use ionic liquid treatment to rapidly disrupt the crystallinity of lignocellolosic biomass substrates. These IL-treated substrates and subjected to dilute acid treatments in a batch (or fed batch) mode for hydrolysis of the cellulose and hemicellulose to sugars.

IL-preprocessed biomass substrates may be subjected to catalytic conversions using mineral acids (e.g., sulfuric acid, hydrochloric acid.) Mild acid hydrolysis according to the present invention is preferably conducted at an acid concentration of 0.01% to 5% by weight (with, for example, either sulfuric acid, hydrochloric acid, phosphoric acid, nitric acid, maleic acid or solid acids) at temperatures ranging from 80-240° C. and pressures ranging from 1 bar to 10 bar. Residence times in the acid treatment is about 1-360 minutes. By varying the acid concentration, residence time and temperature products from hydrolysis reactions such as monomeric sugars or dehydrated products such as HMF and furfural or their subsequent hydrolyzed products levulinic or formic acid are produced. Produce yields and configurations could be modified by controlling the reaction conditions to achieve desired product at moderate operating conditions. IL-treatment of lignocellulosic biomass is conducted as provided in the protocol. U.S. Pat. No. 8,030,030. Utilization of this technology employs moderate temperatures (<200° C.), shorter residence times, and lower pressures to liberate rapid cellulosic sugars in higher yields on IL-treated lignocellulosic biomass substrates. The acid concentration and residence times can be varied to control the desired products (e.g., sugars, HMF, and levulinic acid) and their yields.

The acid may be added to establish a pH of at least about 1, 2, 3, 4, 5, 6, 6.1, 6.2, 6.3, 6.4, 6.5, 6.6, 6.7, 6.8, or 6.9. The acid may be added to establish a pH of at least about between 1-3, 2-4, 3-5, 4-6, or 5-6.5.

The acid may be at least about 1, 2, 3, 4, 5, or 6 M sulfuric acid, hydrochloric acid, nitric acid, or phosphoric acid. The acid may be at least about between 1-3, 2-4, 3-5, 4-6, or 5-6.5 M sulfuric acid, hydrochloric acid, nitric acid, or phosphoric acid.

The acid may be added at least about 1, 2, 3, 4, 5, or 6% sulfuric acid, hydrochloric acid, nitric acid, or phosphoric acid by weight. The acid may be added at least about between 0.01-4%, 0.01-0.05%, 0.05-1%, 0.05-0.075%, 0.1-0.5%, 0.5-2%, 0.01-0.2%, 1-3%, 2-4%, 3-5%, 4-6%, or 5-6.5% sulfuric acid, hydrochloric acid, nitric acid, or phosphoric acid by weight.

The acid hydrolysis may be conducted at least about 50-70° C., 150° C.-200° C., 180° C.-200° C. The acid hydrolysis may be conducted at least about 50° C., 60° C., 70° C., 80° C., 90° C., 100° C., 105° C., 110° C., 120° C., 130° C., 140° C., 150° C., 160° C., 170° C., 180° C., 190° C., or 200° C.

The acid hydrolysis of the conditioned biomass may produce pentose sugars, HMF, or chemicals. The acid hydrolysis methods described herein do not require the addition of enzymes to degrade the cellulosic components of the biomass.

The cellulose may be degraded by the use of cooperative ionic liquid pairs for combined dissolution and catalytic degradation of cellulose into 2-(diethoxymethyl)furan. See Long, et al. (2011) Green Chem. 13: 2334-2338.

Catalysts may be used in the methods described herein to increase the reaction rate of the reactions. For example, alkali and alkaline earth metal chlorides, and transition metal chlorides (e.g., CrCl₃, FeCl₂, and CuCl₂), and IIIA metal chlorides (e.g., AlCl₃) may be used as catalysts. See, e.g., Peng, et al. (2010) Molecules 15: 5258-5272. Additionally, CoSO₄ may be used as a catalyst in conjunction with an ionic liquid.

The sugars produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

The treated biomass produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. Also, the chemical processing of the treated biomass may yield gas productions including but not limited to methane, ethane, CO, CO₂, and H₂.

Homogeneous and heterogeneous catalysis using simple mineral acids and/or supported metal catalysis to transform ionic liquid pre-processed lignocellulosic biomass. Ionic liquid based conditioning of biomass produces uniform substrates amenable to selective catalysis and production of specific products. Catalytic reactions in aqueous media or biphasic systems with good conversions and selectivities, and minimizing costly separations and purifications as is the typical cases with pyrolysis or in ionic mediated catalytic reactions). Multifunctional catalysts with adequate polarity (adsorption properties) and reactant accessibility (porosity) which can work in water or biphasic media with reactants will be used.

Cellulose or biomass hydrolysis in ionic liquid solvent mediated catalysis have resulted in about 50 to 80% glucan conversions from cellulosic components of biomass. In dehydrations reactions, yields of greater than 50% conversion of biomass (or simple sugars) to HMF and furfural could be obtained. Though catalysis in ionic liquid phases have some advantages over conventional catalysis, there are several disadvantages that offset their implementation beyond lab stage for commercial implementation. These are some of the following disadvantages: (i) Ionic liquid mediated catalysis requires reactants (such as biomass, other reactants) dissolved in ionic liquids, (ii) resulting concentrations of dissolved reactants and products are very dilute in concentrations, (iii) difficulty in separation of products, reactants, catalysts and ionic liquids, (iv) ionic liquid phase catalysis is highly sensitive to impurities, (v) recovering and reusing ionic liquids becomes difficult due to product separation issues.

In addition to mineral acids, heterogeneous catalysts such as zeolites and ruthenium may be used for dehydration reactions of IL-preprocessed cellulose to produce HMF. List of catalysts that can be used for production of HMF from IL-treated cellulosic substrates include (i) Organic acids, (ii) Inorganic acids, (iii) metal halides, (iv) salts, (v) Lewis acids, (vi) ion-exchange resins, (vii) zeolites and their combinations. Further, these dehydrated compounds can be hydrogenated to produce DMF and furan based chemicals. Additionally, the sugars produced by the methods described herein may be used to produce succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

The chemical treatment may comprise incubation with acids under heat and pressure or a concentrated acid hydrolysis process (e.g., Scholler process). See also Robinson (1995) “A Mild, Chemical Conversion of Cellulose to Hexane and Other Liquid Hydrocarbon Fuels and Additives,” ACS Fuel Chemistry Preprints 40(3): 729 and Binder & Raines (2010) PNAS 107(10): 4516-4521. The cellulose may be treated with alkaline peroxide and then treated with enzymes to break down the cell wall. For example, the biomass may be treated with an ionic liquid to convert the sugars (e.g., glucose and fructose) into 5-hydroxymethylfurfural (HMF). Oxidation of HMF yields 2,5-furandicarboxylic acid.

In other processes, the cellulose and hemicellulose may be converted to 5-hydroxymethylfurfural (HMF) that may be used as a raw material for plastics and fuels. A metal chloride (e.g., chromium chloride) may be used with an ionic liquid to convert the sugars (e.g., glucose and fructose) into HMF. The chemical, a metal chloride known as chromium chloride, converted sugar into highly pure HMF. The metal chlorides and ionic liquid may be reused. Oxidation of HMF yields 2,5-furandicarboxylic acid, which may be used as a replacement for terephthalic acid in the production of polyesters (e.g., polyethylene terephthalate (PET)). See Zhao, et al. (2007) Science 316(5831): 1597-1600.

Also, the method of acidic hydrolysis of biomass may not include a step of mild alkaline treatment. For example, a method of acidic hydrolysis of biomass may comprise reducing the biomass in size, preferably to particles about 0.1-20 mm in size; ionic liquid (IL) treatment of said biomass; separating the cellulosic, hemicellulosic, and lignin streams; recovering the lignin; adding an acid to the cellulosic stream to lower the pH below pH 7, preferably adding a catalyst; adding an acid to the hemicellulosic stream to lower the pH below pH 7, preferably adding a catalyst; heating the cellulosic stream to heat to a target temperature range, preferably about 150-200° C. for about 15-360 minutes; heating the hemicellulosic stream to heat to a target temperature range, preferably about 150-200° C. for about 15-360 minutes; and recovering chemicals. The chemicals produced by this method may be succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof. The chemicals produced by this method may be 5-hydroxymethyl furfural (HMF), furfural, 2,5-furandicarboxylic acid, formic acid, levulinic acid, or mixtures thereof.

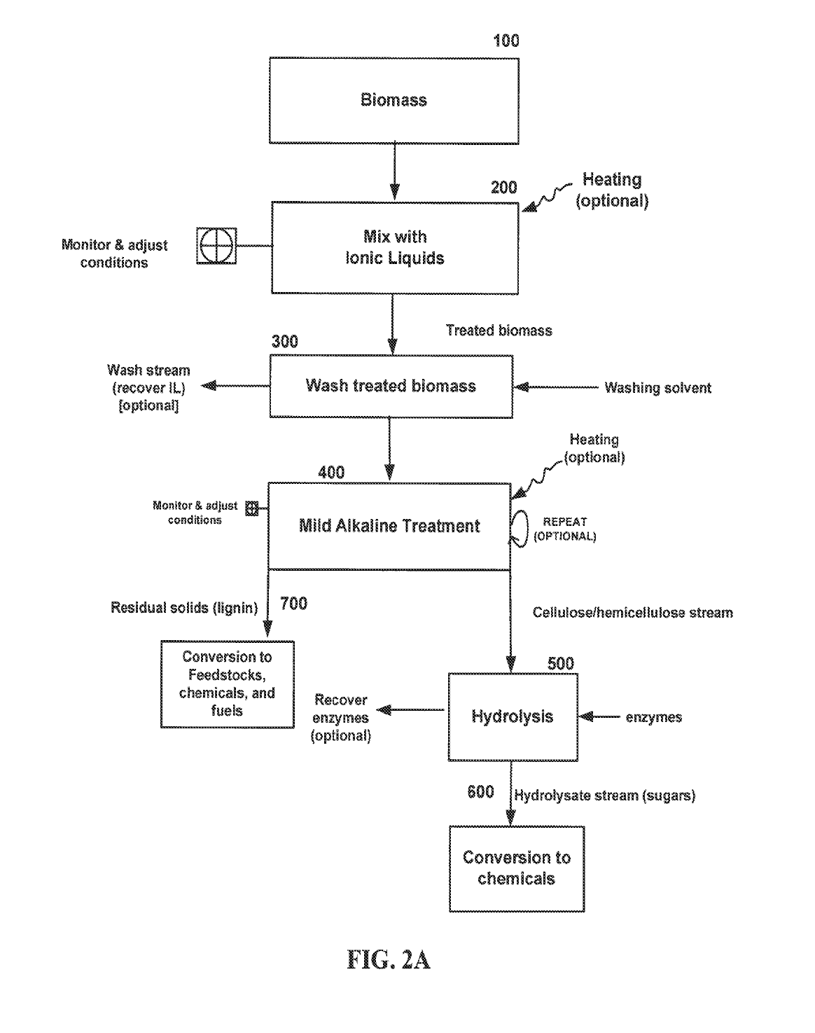

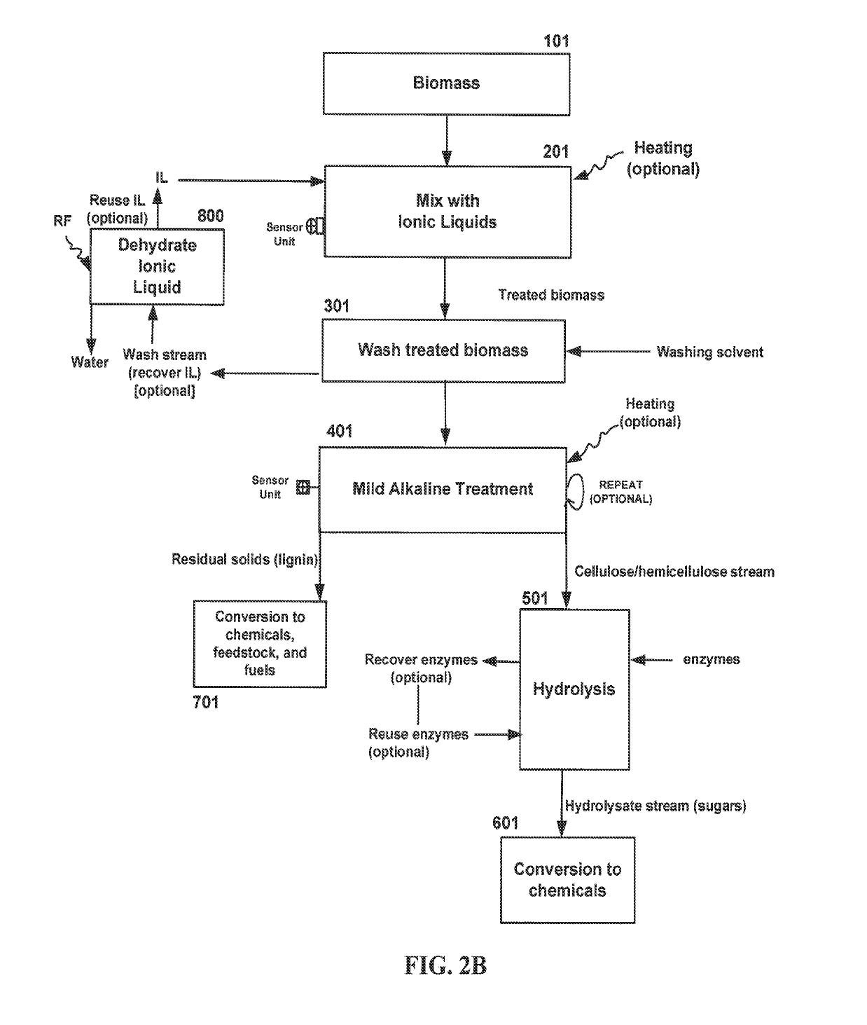

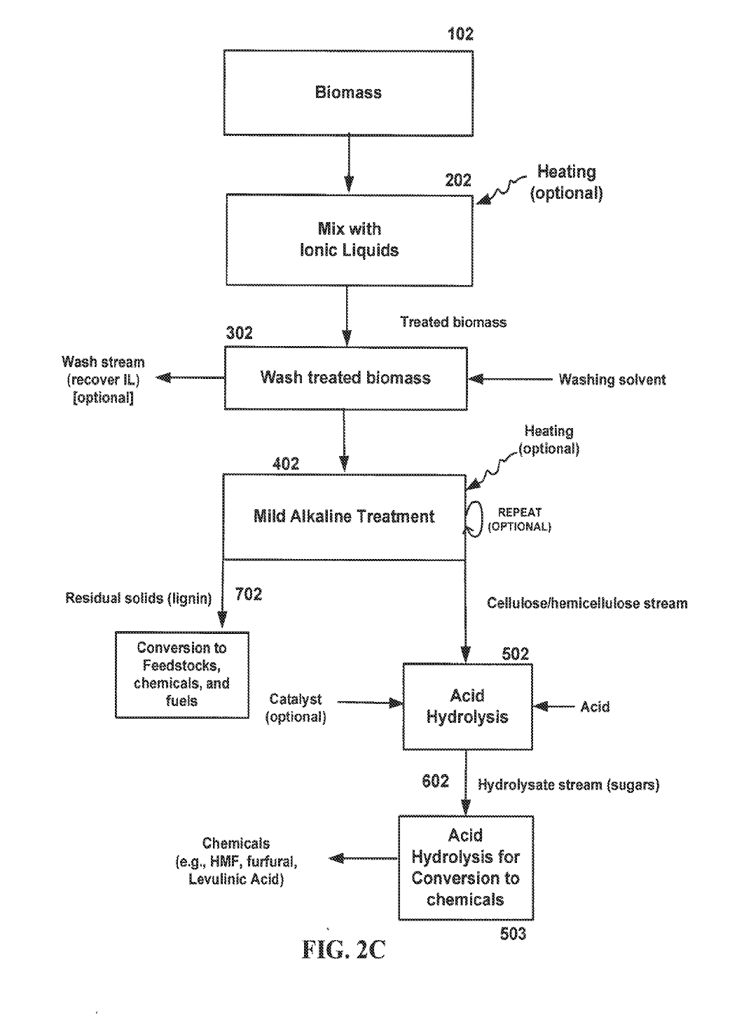

Proceeding now to a description of the drawings, FIG. 2A shows an exemplary series for carrying out steps of a method of the present invention.

One of the following representative ionic liquids 1-n-butyl-3-methylimidazolium chloride (BMIMCl)/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with small particles of biomass 100 (e.g., dry corn stover or poplar (−20+80 mesh sized particles)] for varying times (about 5 minutes to 8 hours) 200. The biomass may be heating using electromagnetic (EM) (e.g., radiofrequency) heating and ultrasonics, electromagnetic (EM) (e.g., radiofrequency), convective, conductive heating, or combinations thereof at about 50° C. to 200° C. as long as the ionic liquid is in molten state during incubation 200. The conditions may be monitored by use of sensors and adjusted to maintain conditions. The biomass may be heated with RF heating at about 27 mHz for at least about 5 seconds to 2 hours. The biomass may then be heated using ultrasonics, electromagnetic (EM) (e.g., radiofrequency), convective, conductive heating, or combinations thereof is for about at least 3-30 minutes or 3-4 hours. The conditions may be monitored and adjusted to maintain uniform heating and sufficient penetration of the biomass by the RF waves.

The treated biomass may then contacted with one of the representative wash-solvents, namely, methanol/ethanol/water/acetonitrile/butanol/propanol 300. The wash-solvent mixes with the IL (in all proportions) and hence is able to extract it from the incubated biomass. The treated biomass may then be separated from the ionic liquid/wash solvent solution by centrifugation. The biomass, stripped off the IL, may then subjected to mild alkaline treatment 400. The IL may be recovered from the wash-solvent and any dissolved biomass components from the wash-step through suitable separation methods including at least one of the following: activated charcoal treatment, distillation, membrane separation, electrochemical separation techniques, solid phase extraction, liquid-liquid extraction, or a combination thereof. The ionic liquid may then be recycled back to the treatment tank. The wash solvent also may be recycled back for reuse in washing IL-incubated biomass. The wash solvent may also be dehydrated by RF heating to dehydrate the wash solvent, driving off the water leaving a dehydrated IL.

The IL may be recovered from the IL/wash solvent mixtures by evaporation of the wash solvent (ethanol and/or water) from the extremely low volatility IL 300. The recovered IL may then be used with no additional cleaning steps in subsequent biomass treatment cycles at constant treatment conditions. The method allows for the repeated reuse of the IL with minimal cleaning which may lead to increased cost savings in IL-treatment.

Residual water in the recycled IL can lower the IL's capacity to sever the inter- and intra-chain hydrogen bonds imparting crystallinity to cellulose. In order to affect swelling of biomass, several of the cellulosic hydrogen-bonds have to be disrupted. Accordingly, it is expect dissolved water to affect IL's performance as a biomass treatment solvent. The admissible water content in IL can affect the economics of the treatment method in two aspects. First, it determines how dry the IL has to be before it can be reused. Second, it determines how dry the biomass has to be during incubation with IL.

In 400, the pretreated biomass is subjected to mild alkaline treatment comprising about 0.5%, 1%, 2%, 3%, 4%, or 5% NaOH or KOH at about a pH of 9-10 for about 30 minutes at 40-50° C. The mild alkaline treatment 400 produces two streams, a lignin rich stream and a cellulose/hemicellulose stream. The lignin may be recovered. The lignin rich steam may be directed to further processing 700 to produce chemicals, feedstocks, and fuels. The cellulose/hemicellulose stream rich stream may be directed to hydrolysis 500 for enzymatic hydrolysis to produce constituent monomers and sugars.

After hydrolysis 500, enzymes may be recovered from the hydrolysis reactor and recycled. Due to the less harsh conditions of the mild alkaline treatment, over 80-90% of the enzymes may be recovered. Further, the enzymatic hydrolysis may be for about 8-12 hours. Complete removal of wash solvent (water) is not necessary before the IL is recycled. Many other treatment methods are not amenable to easy recovery of the chemicals employed in the process. Following hydrolysis (saccharification) 500 with an appropriate enzyme mix, capable of converting all the carbohydrates in the pre-treated biomass to sugars. The resultant hydrolysate stream comprising sugars may be directed to further processing for conversion to chemicals. 600

Most of the solids left behind in the saccharification reactor represent the lignin portion of the biomass. This provides a method of recovering the lignin from biomass 700 Also, ultra-filtration of the liquid portion of the hydrolysate, provides a means of recovering the hydrolysis enzymes for reuse from the sugar solution which is the precursor for the production of a number of fuels and feedstock 700.

Steps 200, 300, and/or 400 may be repeated. Further, steps 200 and/or 400 may be carried out in batch or continuous form. The goal of treatment 200 is not achieving any dissolution of lignocellulose, but treatment of the pretreated biomass for sufficient time to release lignin and swell the remaining biomass structure to enhance the hydrolysis rate and conversion of cellulose and hemicellulose to their constituent sugars 500.

FIG. 2B shows an exemplary series for carrying out steps of a method of the present invention.

One of the following representative ionic liquids 1-n-butyl-3-methylimidazolium chloride (BMIMCl)/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with small particles of biomass 101 (e.g., dry corn stover or poplar (−20+80 mesh sized particles)] for varying times (about 5 minutes to 8 hours) to form a biomass 201. Heating of the biomass may be carried out by first electromagnetic (EM) (e.g., radiofrequency) heating to reach a target temperature or temperature range (e.g., 50° C.-220° C.) 201 and then heating using ultrasonics, electromagnetic (EM) (e.g., radiofrequency), convective, conductive heating, or combinations thereof at about 50° C. to 200° C. for 3-30 minutes or 3-4 hours 201.

The treated biomass may then contacted with one of the representative wash-solvents, namely, methanol/ethanol/water/acetonitrile/butanol/propanol 301. The wash-solvent mixes with the IL (in all proportions) and hence is able to extract it from the incubated biomass. The treated biomass may then be separated from the ionic liquid/wash solvent solution by centrifugation. The biomass, stripped off the IL, may then hydrolyzed with a cellulase system 501.

The IL may be recovered from the wash-solvent and any dissolved biomass components from the wash-step through suitable separation methods including at least one of the following: activated charcoal treatment, distillation, membrane separation, electrochemical separation techniques, solid phase extraction, liquid-liquid extraction, or a combination thereof. The ionic liquid may then be recycled back to the treatment tank. The wash solvent also may be recycled back for reuse in washing IL-incubated biomass. The IL may be recovered from the IL/wash solvent mixtures by evaporation of the wash solvent (ethanol and/or water) from the extremely low volatility IL 301. The wash solvent may also be dehydrated by RF heating to dehydrate the wash solvent, driving off the water leaving a dehydrated IL 800. The recovered IL may then be used with no additional cleaning steps in subsequent biomass treatment cycles at constant treatment conditions. The method allows for the repeated reuse of the IL with minimal cleaning which may lead to increased cost savings in IL-treatment.

In 401, the pretreated biomass is subjected to mild alkaline treatment comprising about 0.5%, 1%, 2%, 3%, 4%, or 5% NaOH or KOH at about a pH of 9-10 for about 30 minutes at 40-50° C. The mild alkaline treatment 401 produces two streams, a lignin rich stream and a cellulose/hemicellulose stream.

The lignin rich steam may be directed to further processing 701 to produce chemicals, feedstocks, and fuels.

The cellulose/hemicellulose stream rich stream may be directed to hydrolysis 501 for enzymatic hydrolysis to produce constituent monomers and sugars.

After hydrolysis 501, enzymes may be recovered from the hydrolysis reactor and recycled. Complete removal of wash solvent (water) is not necessary before the IL is recycled. Many other treatment methods are not amenable to easy recovery of the chemicals employed in the process. Following hydrolysis (saccharification) 501 with an appropriate enzyme mix, capable of converting all the carbohydrates in the pre-treated biomass to sugars. The resultant hydrolysate stream comprising sugars may be directed to further processing for conversion to chemicals 601.

Most of the solids left behind in the saccharification reactor represent the lignin portion of the biomass. This provides a method of recovering the lignin from biomass 701. Also, ultra-filtration of the liquid portion of the hydrolysate, provides a means of recovering the hydrolysis enzymes for reuse from the sugar solution which is the precursor for the production of a number of fuels and feedstock 701.

The conditions may be monitored by use of sensors and adjusted to maintain conditions 201. The conditions may be monitored and adjusted to maintain uniform heating and sufficient penetration of the biomass by the RF waves. Steps 201, 301, and/or 401 may be repeated. Further, steps 201, 301, and/or 401 may be carried out in batch or continuous form.

FIG. 2C shows an exemplary series for carrying out steps of a method of the present invention.

One of the following representative ionic liquids 1-n-butyl-3-methylimidazolium chloride (BMIMCl)/1-n-ethyl-3-methyl imidazolium acetate (EMIMAc)/1-ethyl-3-methyl imidazolium propionate (EMIMPr)/1-allyl-3-methyl imidazolium chloride/3-methyl-N-butylpyridinium chloride may be contacted with biomass 102 (e.g., dry corn stover or poplar] for varying times (e.g., about 5 minutes to 8 hours) to swell but not dissolve biomass 202 (e.g., about 30%). Heating of the biomass may be carried out by first electromagnetic (EM) (e.g., radiofrequency) heating to reach a target temperature or temperature range (e.g., 50° C.-220° C.) 202 and then heating using ultrasonics, electromagnetic (EM) (e.g., radiofrequency), convective, conductive heating, or combinations thereof at about 50° C. to 200° C. for 3-30 minutes or 3-4 hours 202.

The treated biomass may then contacted with one of the representative wash-solvents, namely, methanol/ethanol/water/acetonitrile/butanol/propanol 302. The wash-solvent mixes with the IL (in all proportions) and hence is able to extract it from the incubated biomass. The treated biomass may then be separated from the ionic liquid/wash solvent solution by centrifugation. The cellulose and hemicellulose may then underdo acid hydrolysis system 502.

In 402, the pretreated biomass is subjected to mild alkaline treatment comprising about 0.5%, 1%, 2%, 3%, 4%, or 5% NaOH or KOH at about a pH of 9-10 for about 30 minutes at 40-50° C. The mild alkaline treatment 402 produces two streams, a lignin rich stream and a cellulose/hemicellulose stream.

The lignin may be recovered to further processing 702 to produce chemicals, feedstocks, and fuels.

The cellulose/hemicellulose stream rich stream may be directed to acid hydrolysis 502 for enzymatic hydrolysis to produce constituent monomers and sugars.

Following acid hydrolysis (saccharification) 502 with an appropriate acids, optionally with a catalyst, capable of converting the cellulose and hemicellulose to sugars 602. The resultant hydrolysate stream comprising sugars may be directed to further processing for conversion to chemicals by acid hydrolysis, optionally with a catalyst 503.

The conditions may be monitored by use of sensors and adjusted to maintain conditions 201. The conditions may be monitored and adjusted to maintain uniform heating and sufficient penetration of the biomass by the RF waves. Steps 202, 402, 502, and/or 602 may be repeated. Further, steps 202, 302, 402, 502, and/or 602 may be carried out in batch or continuous form.

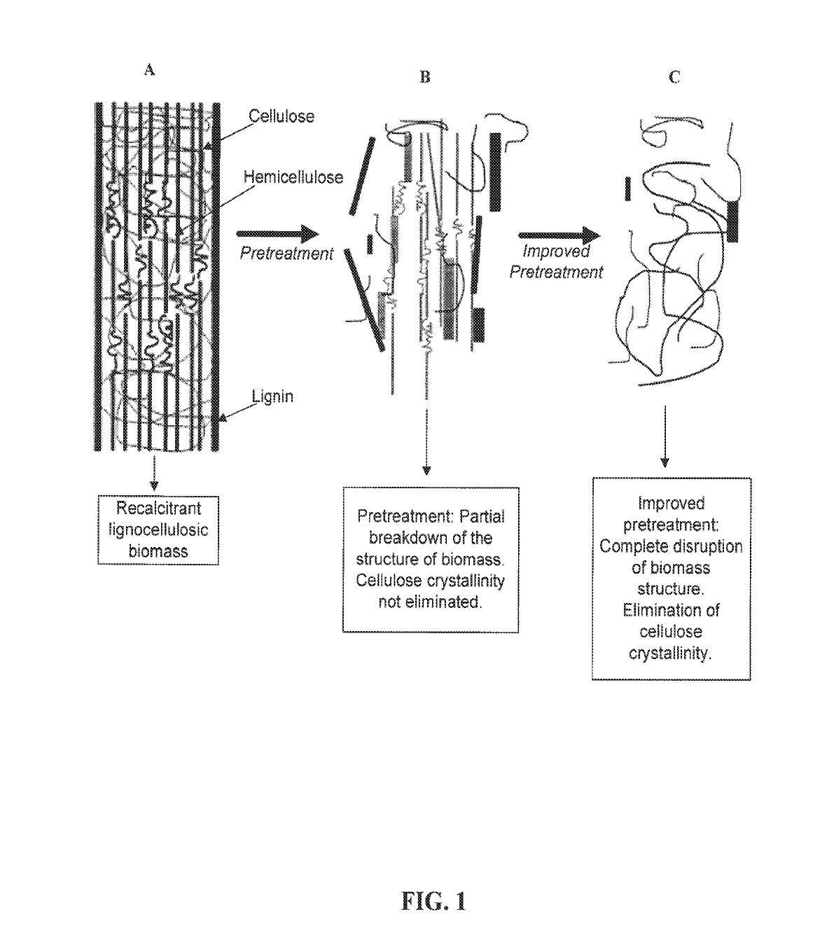

After enzyme hydrolysis 500 and 501, the enzymes may be reclaimed. The inventors surprisingly discovered that the use of mild alkaline treatment conditions allows for the recovery of a high percentage of enzymes (e.g., >90%) and their reuse in more cycles than prior methods (e.g., 16-20 cycles versus 1 cycle of hydrolysis). The inventor surprisingly discovered that ionic liquid pretreatment disrupts the crystallinity of cellulose rapidly for effective hydrolysis. However, the inventors found that the ionic liquid pretreatment results in the swelling of the biomass and does not remove the lignin cladding, though disrupting it. See FIG. 1. Therefore, this remaining lignin can act as a physical barrier for enzymatic hydrolysis of cellulosic and hemicellulosic sugars as it sheaths and covers it, slowing enzymatic hydrolysis (e.g., preventing the enzymes from reaching the cellulose and hemicellulose). Lignin also binds to the enzymes reducing their activity and further reducing the ability to recover and reuse the enzymes. Therefore, removal of lignin from swollen biomass can enhance hydrolysis rates by providing more direct access to cellulosic chains and severely reducing the scope of enzyme loss due to lignin binding.

The current method of combining ionic liquid pretreatment with mild alkaline processing benefits from either alone, or the usually more harsh alkaline treatments used in the art. For example, optimal lime pretreatment conditions were ascertained to be 4 weeks aeration of biomass with lime (1 gram of lime per gram of raw biomass at 55° C. in 91% glucan and 51% xylan yields. See “Lime Pretreatment and Enzymatic Hydrolysis of Corn Stover.” A Dissertation by Se Hoon Kim (2004) Texas A&M University. This is in contrast with over 90% glucan yield and over 60% xylan yield after ionic liquid pretreatment plus mild alkaline treatment at 75° C. for 60 minutes. Thus, the methods described herein reaches the same yields as a conventional lime pretreatment in a matter of minutes rather than weeks.

Further, thermophilic enzymes may be used in the hydrolysis step (e.g., enzymes stable and active at about 70° C.). The use of thermophilic enzymes allows for the hydrolysis step to be run at a higher temperature and improves efficiency and yield of the hydrolysis step. For example, mixtures of thermophilic endo- and exo-glycoside hydrolases may be active at high temperatures and acidic pH. The thermophilic enzymes may be isolated from thermophilic bacteria including but not limited to Sulfolobus solfataricus, Alicyclobacillus acidocaldarius, and Thermus thermophilus. Also, thermophilic cellulases may be used.

Although certain manufacturers, model names and numbers are given for machinery used in the invention, other machinery may be substituted, as would be appreciated by those skilled in the art.

Although certain ranges are provided for the temperature, conveyor speed, electromagnetic (EM) (e.g., radiofrequency) wave intensity, and pressure characteristics, these can be varied based on the particular volumes desired, space requirements and other needs. After reading this specification, one skilled in the art will understand that the selection of working or optimum numbers for these variables may be made once the plant and overall process parameters of a particular processing installation are known.

Additionally, although preferred systems are disclosed for controlling the temperature of the biomass, these may be varied. These may be varied by substituting, depending on normal plant considerations of energy cost, plant lay-out and the like, and generally the temperature values used in the process tolerate some ongoing variability due to, for instance, changes in ambient plant temperatures and other related factors.

All publications (e.g., Non-Patent Literature), patents, patent application publications, and patent applications mentioned in this specification are indicative of the level of skill of those skilled in the art to which this invention pertains. All such publications (e.g., Non-Patent Literature), patents, patent application publications, and patent applications are herein incorporated by reference to the same extent as if each individual publication, patent, patent application publication, or patent application was specifically and individually indicated to be incorporated by reference.

Although methods and materials similar or equivalent to those described herein may be used in the invention or testing of the present invention, suitable methods and materials are described herein. The materials, methods and examples are illustrative only, and are not intended to be limiting.

The invention now being generally described, it will be more readily understood by reference to the following examples, which are included merely for purposes of illustration of certain aspects and embodiments of the present invention, and are not intended to limit the invention.

### EXAMPLES

### Example 1

### Treatment of Wheatstraw

Wheatstraw was comminuted and then subjected to 25% ionic liquid pretreatment for about 2.15 hours. The pretreated wheatstraw was then divided in two groups, the first group then underwent no further treatment before about 2.5 hours of enzymatic hydrolysis and the second group underwent mild alkaline treatment at 5% sodium hydroxide for about 1 hour at about 75° C. The results are shown in Table 1.

TABLE 1
Wheatstraw IL pretreatment with and without mild alkaline treatment.

| | | | | Hydrolysis Time | | Hydrolysis Time | |
|---|---|---|---|---|---|---|---|
| | Ionic liquid | | | (hours) | | (hours) | |
| | pretreatment | Caustic | % | 6 | | 13 | |
| Biomass | Condition | Conditions | Enzymes | % Glucan | % Xylan | % Glucan | % Xylan |
| Wheatstraw | 25% PT WS | None | 2.5 | 44 | 26 | 49 | 30 |
| | 2.15 hours | | | | | | |
| Wheatstraw | 25% PT WS | 75° C. 1 hour, | 2.5 | 81 | 36 | 84 | 38 |
| | 2.15 hours | 5% Treat | | | | | |

The combination of the ionic liquid pretreatment and mild alkaline treatment surprisingly improved the yield of glucan and xylan from wheatstraw (lignocellulosic biomass) in a shorter period of time. Thus, the combination of the ionic liquid pretreatment and mild alkaline treatment may be used to improve the enzymatic hydrolysis of the treated biomass in a shorter period of time than prior art methods.

### Example 2

### Comparison of Wheatstraw Treatment with and Without Pretreatment and/or Alkaline Treatment

Wheatstraw was comminuted and then divided into two groups. One group of wheatstraw was subjected to 25% ionic liquid pretreatment for about 2.15 hours and the other received no pretreatment. The pretreated and not-pretreated wheatstraw was then divided in two groups, the first group then underwent mild alkaline treatment with sodium hydroxide for about 1 hour at about 75° C. for about 15 minutes at about 50° C. All of the groups underwent either × enzyme or 2× enzyme hydrolysis. The results are shown in Table 2.

TABLE 2
Wheatstraw Processing with and without IL pretreatment and/or mild
alkaline treatment.

| | | | | | Hydrolysis | | Hydrolysis | | Hydrolysis | |
|---|---|---|---|---|---|---|---|---|---|---|
| | | | | | Time (hours) | | Time (hours) | | Time (hours) | |
| | Ionic | | | | 6 | | 13 | | 24 | |
| | liquid | | | % | % | % | % | % | % | % |
| Biomass | PT? | Conditioning | Enzymes | Hydrolysis | Glucan | Xylan | Glucan | Xylan | Glucan | Xylan |
| Wheatstraw | Yes | 75° C. 1 hour | x | 5.5 | 70 | 48 | 90 | 62 | 96 | 67 |
| Wheatstraw | Yes | 75° C. 1 hour | x | 5.5 | 75 | 51 | 95 | 65 | | |
| Wheatstraw | Yes | 50° C., 15 min. | 2x | 5.5 | 84 | 69 | 98 | 79 | 103 | 86 |
| Wheatstraw | Yes | None | x | 5.5 | 53 | 50 | 69 | 60 | 75 | 60 |
| Wheatstraw | No | 75° C. 1 hour | x | 5.5 | 15 | 7 | 19 | 9 | 22 | 11 |
| Wheatstraw | No | None | x | 5.5 | 6 | 3 | 9 | 4 | 10 | 6 |

The combination of the ionic liquid pretreatment and mild alkaline treatment surprisingly improved the yield of glucan and xylan from wheatstraw (lignocellulosic biomass) in a shorter period of time. As seen in Table 2, the lack of either IL pretreatment or mild alkaline treatment reduces the yield of sugars (e.g., glucan, xylan) from hydrolysis. Thus, the combination of the ionic liquid pretreatment and mild alkaline treatment may be used to improve the enzymatic hydrolysis of the treated biomass in a shorter period of time than prior art methods.

### Example 3

### Comparison of Wheatstraw Treatment with and Without Pretreatment and/or Alkaline Treatment

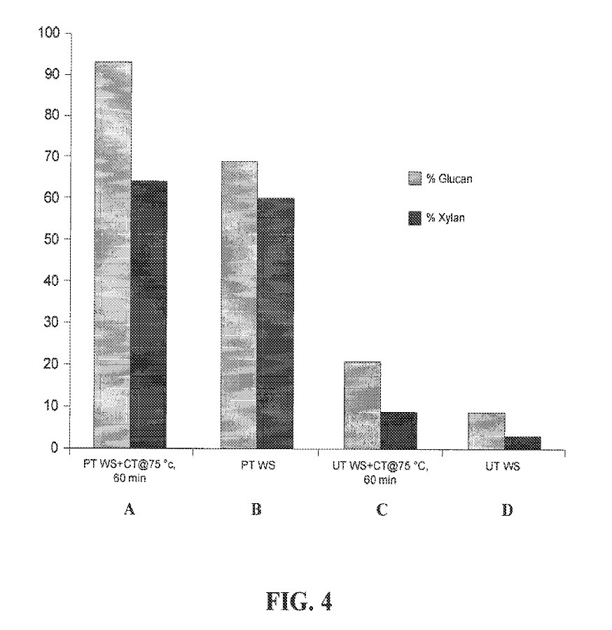

Enzymatic hydrolysis results of wheat straw biomass after 13 hours of hydrolysis at 2× Enzyme dosage. Four conditions were compared: (1) Ionic liquid pretreatment of wheatstraw with mild alkaline processing at 75° C. 1 hour, (2) Ionic liquid pretreatment of wheatstraw alone, (3) mild alkaline processing at 75° C. 1 hour with no ionic liquid pretreatment and (4) untreated wheat straw. These four conditions were compared for the percentage of glycan and xylan conversion to monomeric sugars. See FIG. 4. Thus, either the ionic liquid pretreatment or mild alkaline treatment may improve the yield of monomeric sugars. However, the combination of both the ionic liquid pretreatment and mild alkaline treatment showed a greater than additive effect in the yield of monomeric sugars from hydrolysis. Further, the pretreatment and mild alkaline treatment processing time is measured in a matter of minutes to hours instead of days to weeks with prior methods.

### Example 4

### Comparison of Wheatstraw Treatment with and Without Pretreatment and/or Alkaline Treatment

Wheatstraw was comminuted and then divided into different groups that compared ionic liquid pretreatment in combination with various alkaline agents for the yield from enzymatic hydrolysis. All of the groups underwent enzyme hydrolysis and the amount of glucan and xylan after 12 hours and 24 hours was examined. The results are shown in Table 3.

TABLE 3

| | | | | | % Alkali | | | Enzymatic Hydrolysis | | | |
|---|---|---|---|---|---|---|---|---|---|---|---|
| | | | | Caustic | (w/w) | | | 12 hrs | | 24 hrs | |
| | | Ionic Liquid | | Conditions - | (w.r.t. Dry | Enzyme | Temp | % | % | % | % |
| Sample | Biomass | Pretreatment | Caustic | time, temp | Biomass) | Loading | (° C.) | Glucan | Xylan | Glucan | Xylan |
| 1 | Wheat Straw | Yes | None | | 0.00% | 0 | 0 | 61% | 51% | 71% | 59% |
| 2 | Wheat Straw | Yes | NaOH | 60 min, 50 C. | 16.65% | 2% | 50 | 96% | 54% | 99% | 62% |
| 3 | Wheat Straw | Yes | NaOH | 60 min, 50 C. | 2.20% | 2% | 50 | 73% | 62% | 80% | 68% |
| 4 | Wheat Straw | Yes | NaOH | 60 min, 50 C. | 8.75% | 2% | 50 | 81% | 60% | 89% | 67% |
| 4 | Wheat Straw | No | NaOH | 60 min, 50 C. | 8.75% | 2% | 50 | 33% | 35% | 40% | 38% |
| 5 | Wheat Straw | Yes | KOH | 60 min, 50 C. | 8.75% | 2% | 50 | 84% | 69% | 92% | 77% |
| 5 | Wheat Straw | No | KOH | 60 min, 50 C. | 8.75% | 2% | 50 | 27% | 21% | 33% | 23% |
| 6 | Wheat Straw | Yes | Ca(OH)₂ | 60 min, 50 C. | 8.75% | 2% | 50 | 91% | 78% | 98% | 91% |
| 6 | Wheat Straw | No | Ca(OH)₂ | 60 min, 50 C. | 8.75% | 2% | 50 | 31% | 27% | 36% | 31% |
| 7 | Wheat Straw | Yes | Mg(OH)₂ | 60 min, 50 C. | 8.75% | 2% | 50 | 62% | 53% | 66% | 58% |
| 8 | Wheat Straw | Yes | Al(OH)₃ | 60 min, 50 C. | 8.75% | 2% | 50 | 68% | 56% | 74% | 61% |

The combination of the ionic liquid pretreatment and mild alkaline treatment surprisingly improved the yield of glucan and xylan from wheatstraw (lignocellulosic biomass) in a shorter period of time. As seen in Table 3, the lack of either IL pretreatment or mild alkaline treatment reduces the yield of sugars (e.g., glucan, xylan) from hydrolysis. Thus, the combination of the ionic liquid pretreatment and mild alkaline treatment may be used to improve the enzymatic hydrolysis of the treated biomass in a shorter period of time than prior art methods.

### Example 5

### Mild Alkaline Treatment of Biomass to Produce Glucan

Mild alkaline conditioned of IL-conditioned lignocellulosic biomass substrates enables the efficient removal of lignin and rapid enzymatic digestibility (or acid hydrolysis) of subsequent carbohydrate substrates.

Table 4 show enhanced enzymatic digestibility of mild alkaline conditioned IL-treated biomass substrates. Significant improvements in glucan conversion were observed for enzymatic hydrolysis of mild alkaline conditioned IL-treated substrates within 12 hours of hydrolysis.

TABLE 4
Improvement in Percent Glucan conversion observed
from 12 hour enzymatic hydrolysis of Ionic Liquid
Treated Substrates after caustic conditioning.

| | IL | | % Glucan |
|---|---|---|---|
| Feed Stock | Treatment | Caustic Condition | (12 hr) |
| Poplar | Yes | None | 63 |
| Poplar | Yes | 0.25% (w/v) NaOH | 98 |
| | | 70° C. for 1 hour | |
| Wheat Straw | Yes | None | 45 |
| Wheat Straw | Yes | 0.25% (w/v) NaOH | 77 |
| | | 70° C. for 1 hour | |

The methods used herein to pretreat the biomass are conducted at mild alkaline (<0.5% w/v), mild temperature (<80° C.) and time (<1 hr) unlike regular alkaline treatments which are conducted at elevated temperatures or at lower temperatures for several days. During mild alkaline treatment on IL-conditioned biomass, lignin gets separated from the biomass leaving cellulose and hemicellulose for rapid hydrolysis. In soda or Kraft processes and alkaline pretreatments, which were conducted at higher temperatures (e.g., above 120° C. to 180° C.), lignin condensation reactions occur, whereas at mild alkaline conditioning on IL-treated biomass, lignin removal without the formation of condensation products are possible. In mild alkaline conditioning on IL-conditioned biomass, only lignin gets separated whereas at higher alkaline treatments on native biomass substrates, significant amount of hemicellulose is also lost along with lignin removal and degradation. Thus the methods described herein allow for the separation and recovery of lignin for further processing into chemicals without the loss of hemicellulose.

### Example 6

### Acid Hydrolysis of Biomass

Acid hydrolysis of biomass after ionic liquid pretreatment as described herein increases the yield of pentoses (e.g., glucan) and chemicals.

TABLE 5
Acid Hydrolysis of cellulosic biomass components of Untreated & Ionic
Liquid treated substrates at 150-180° C. for 1 hour with 0.01-4%
dilute H₂SO₄

% Glucan

| Biomass | Untreated | IL Treated |
|---|---|---|
| Avicel | 12 | 48 |
| Poplar | 20 | 68 |
| Poplar | 21 | 75 |
| Corn Stover | 15 | 45 |

TABLE 6
Acid Hydrolysis of C₆ sugars of Untreated & Ionic Liquid treated
substrates at 150-180° C. for 1 hour with 0.02-4% dilute sulfuric acid

% Glucan

| Biomass | Untreated | IL Treated |
|---|---|---|
| Avicel | 12 | 48 |
| Poplar | 21 | 68 |
| Poplar | 20 | 75 |
| Corn Stover | 15 | 45 |

TABLE 7
Yields of hemicellulose sugar (xylose) from dilute acid (0.01-4%) of
hydrolysis of various lignocellulosic biomass substrates at 150° C.

| Biomass | Time (min) | % Xylose |
|---|---|---|
| Corn Stover | 15 | 86 |
| Ash | 30 | 82 |
| Poplar | 45 | 71 |

TABLE 8
Acid Hydrolysis of cellulosic biomass components of Untreated & IL
treated substrates at 150-200° C. for 15-360 minutes with
0.01-4% dilute sulfuric acid (H₂SO₄).

% Glucan

| Biomass | Untreated | IL Treated |
|---|---|---|
| Avicel | 12 | 48 |
| Poplar | 20 | 75 |
| Poplar | 20 | 80 |
| Corn Stover | 15 | 45 |
| Ash | 10 | 68 |

Thus, the acid hydrolysis methods described herein provided an unexpected improvement in the yield of glucan and xylan (sugars) in ionic liquid conditioned versus unconditioned biomass.

### Example 7

### Direct Cellulose Conversion to HMF

Dilute acid catalysis of ionic liquid conditioned lignocellulosic substrates produced high glucose yields. Without such conditioning, only concentrated acid (acid concentration greater than 40%) catalysis can produce high yields of glucose. Dilute acid catalysis of ionic liquid conditioned cellulosic substrates generates HMF in high yield in a single phase aqueous solution containing dilute acid (or zeolite catalyst) and water at moderate conditions, without significant degradation products. Other known process converting cellulose to HMF require solvent extraction of product to avoid generation of degradation products.

TABLE 9
Acid and zeolite catalysis of IL-treated corn stover for
production of HMF from cellulosic component of
IL-treated corn stover at 150-200° C.

| Biomass | Catalyst | HMF |
|---|---|---|
| Corn stover | 4% acid | 77% |
| Corn stover | 4% acid | 56% |
| Corn stover | zeolite | 82% |

Thus, the combination of mild acidic treatment and a catalyst provided an unexpected improvement in the yield of HMF as compared to prior art methods.

### Example 8

### Direct Cellulose Conversion to Levulinic Acid

Dilute acid catalysis of ionic liquid conditioned cellulosic substrates generates levulinic acid and formic acid in high yields at moderate temperatures and pressures. Production of sugar alcohols, polyols such as ethylene glycol, may be produced directly from cellulose after ionic liquid biomass conditioning described herein. Levulinic acid is produced from IL-preprocessed cellulosic substrates via aqueous phase acid catalytic reactions.

TABLE 10
Acid catalysis of cellulosic components to produce Levulinic acid
under mild acidic conditions (4% acid 1 hour at 180° C.)

| Feed Stock | % Glucose | % Levulinic Acid |
|---|---|---|
| Avicel | 37 | 50 |
| (Ionic Liquid | | |
| pretreated) | | |

Thus, the use of a catalyst provided to produce levulinic acid under mild acidic conditions an unexpected improvement in the yield as compared to prior art methods.

### Example 9

### Catalytic Conversion of Ionic Liquid Preprocessed Cellulose for Polyols Production (Ethylene Glycol)

Heterogeneous catalysts can be successfully applied to facilitate IL-preprocessed cellulose depolymerization in hydrothermal conditions at milder conditions. IL-preprocessing renders the cellulose chains highly amenable to catalytic conversions for production of sugars alcohols and ethylene glycol. To augment the hydrolysis of cellulose, solid or liquid acids can be introduced into the reaction, such as heteropolyacids. In this process, firstly, cellulose was hydrolyzed to cellooligosaccharides. Then, cellooligosaccharides are gradually converted into glycolaldehyde through retro-aldol reaction. Finally, the intermediate glycolaldehyde can be instantly hydrogenated to ethylene glycol over the hydrogenation catalysts. Hydrogenolysis of IL preprocessed cellulose to C₄-C₇ alcohols can be conducted in methanol using simple catalysts such as CuO/Al₂O₃ which can be used as fuels and fuel replacements. The use of a catalyst provided to produce sugars alcohols and ethylene glycol under mild acidic conditions an unexpected improvement in the yield as compared to prior art methods.

Those skilled in the art will recognize, or be able to ascertain using no more than routine experimentation, many equivalents to the specific embodiments of the invention described herein. Such equivalents are intended to be encompassed by the following claims.

We claim:

1. A method for the treatment of lignocellulosic biomass comprising
(a) mixing lignocellulosic biomass with an ionic liquid for a sufficient time and temperature to swell the lignocellulosic biomass without dissolution of the lignocellulosic biomass in the ionic liquid; and
(b) treating the swelled lignocellulosic biomass under mild alkaline treatment to separate the lignin from the cellulose and hemicellulose.

2. The method of claim 1, wherein said mild alkaline treatment comprises the addition of an alkaline agent.

3. The method of claim 2, wherein said alkaline agent is NaOH, aqueous ammonia, LiOH, Mg(OH)₂, Al(OH)₃, Ca(OH)₂, H₂O₂, NaS, Na₂CO₃, or a combination thereof.

4. The method of claim 2, wherein said alkaline agent is added at about 0.1, 0.2, 0.25, 0.3, 0.4, 0.5, 0.6, 0.7, 0.8, 0.9, 1, 2, 2.1, 2.2, 2.3, 2.4, 2.5, 3, 4, 5, 6, 7, 8, 8.25, 8.5, 8.75, 9, 10, 11, 12, 13, 14, 15, 16, 16.2, 16.4, 16.5, 16.65, 16.7, 16.8, 17, 18, 19, or 20% by weight.

5. The method of claim 2, wherein said alkaline agent is added at about 0.1-10%, 0.1-0.5%, 0.1-5%, 2-8%, 5-15%, 15-20%, 10-20% by weight.

6. The method of claim 2, wherein said mild alkaline condition comprise a pH of about 8-11, pH 8-10, pH 9-11, pH 9-10, pH 10-11, pH 9.5-10.5, 8, 8.25, 8.5, 8.75, 9, 9.25, 9.5, 9.75, 10, 10.25, 10.5, 10.75, 11, 11.25, 11.5, 12, 12.25, 12.5, or 13.

7. The method of any one of claims 2-6, wherein said alkaline treatment is at a temperature of at least about 10° C., 20° C., 30° C., 40° C., 50° C., 60° C., 70° C., 73° C., 75° C., 78° C., or 80° C.

8. The method of any one of claims 2-6, wherein said alkaline treatment is at a temperature of at least about 10-50° C., 30-70° C., 40° C.-60° C., 50° C.-70° C., 50° C.-80° C., 40° C.-80° C., 50-80° C., 50° C.-70° C., 50° C.-60° C., or 40° C.-70° C.

9. The method of any one of claims 2-6, wherein said mild alkaline treatment is for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 minutes.

10. The method of any one of claims 2-6, wherein said mild alkaline treatment is for about 1-60, 1-70, 1-75, 1-80, 1-30, 1-20, 5-10, or 1-15 minutes.

11. The method of any one of claims 1-10, wherein said method further comprises hydrolysis of the cellulose and hemicellulose with a biochemical reagent to convert the cellulose and hemicellulose to sugars.

12. The method of claim 11, wherein said sugars are hexose and/or pentose sugars.

13. The method of claim 11, wherein said biochemical reagent is an enzyme.

14. The method of claim 13, wherein said enzyme is recovered.

15. The method of claim 14, wherein at least 90, 91, 92, 93, 94, 95, 96, 97, 98, or 99% of the enzyme is recovered.

16. The method of claim 15, wherein said enzyme is reused.

17. The method of claim 16, wherein said enzyme is reused for about 1-20 hydrolysis cycles.

18. The method of claim 17, wherein said enzyme is reused for about 1, 2, 3, 4, 5, 6, 7, 8, 9, 10, 11, 12, 13, 14, 15, 16, 17, 18, 19, or 20 hydrolysis cycles.

19. The method of claim 17, wherein said enzyme is reused for about 16-20 hydrolysis cycles.

20. The method of any one of claims 11-19, wherein the biomass is heated to about 51° C., 52° C., 53° C., 54° C., 55° C., 56° C., 57° C., 58° C., 59° C., 60° C. 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

21. The method of any one of claims 11-20, wherein method further comprises acid hydrolysis of the sugars to produce chemicals.

22. The method of any one of claims 1-21, wherein said method further comprises acid hydrolysis of the cellulose and hemicellulose to convert the cellulose and hemicellulose to sugars, chemicals, or combinations thereof.

23. The method of claim 22, wherein said acid is phosphoric acid, nitric acid, maleic acid, solid acids, sulfuric acid, hydrochloric acid, or a combination thereof.

24. The method of claim 23, wherein said solid acid is sulphamic acid, citric acid, oxalix acid, benzoic acid, CsHSO₄, CsHSeO₄, or a combination thereof.

25. The method any one of claims 21-24, wherein said acid hydrolysis is at a pH of about 1, 2, 3, 3.5, 4, 4.5, 5, 5.5, 5.8, 6, 6.5, 6.8, 1-3, 2-4, 3-5, 2-6, 3.5-4.5, or 4-6.

26. The method any one of claims 21-24, wherein said acid is added at acid concentration of at least about 0.01-1%, 0.1-2%, 0.1-4%, 0.2-4%, 0.05-5%, 0.01-5%, 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 2%, 3%, 4%, or 5% by weight.

27. The method any one of claims 21-24, wherein said acid hydrolysis is at a temperature of at least about 80° C., 80° C.-200° C., 150° C.-180° C., 80° C.-240° C. 150° C.-160° C., 140° C.-170° C., 140° C., 150° C., 170° C., 180° C., 140° C.-160° C., 150° C.-170° C., 150° C.-180° C., or 140° C.-200° C.

28. The method any one of claims 21-24, wherein said acid hydrolysis is for about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 110, 120, 130, 140, 150, 160, 170, 180, 190, 200, 210, 220, 230, 240, 250, 260, 270, 280, 290, 1-60, 1-80, 1-100, 1-120, 1-180, 1-200, 1-300, 1-340, or 1-360 minutes.

29. The method of any one of claims 21-28, wherein said acid hydrolysis is at a pressure of at least about 100-1,000 kPa.

30. The method of any one of claims 21-29, wherein said acid hydrolysis further comprises adding a catalyst.

31. The method of claim 30, wherein said catalyst a metal halide, oxide, multifunctional homogenous catalyst, multifunctional heterogenous catalyst, resin, salt, zeolite, or a combination thereof.

32. The method of claim 31, wherein said metal halide is a metal fluoride, metal chloride, metal bromide, metal iodide, metal astatide, or combinations thereof.

33. The method of claim 32, wherein said zeolite is analcime, chabazite, clinoptilolite, heulandite, natrolite, phillipsite, thromsonite, stilbite, gonnardite, natrolite, mesolite, paranatrolite, scolecite, tetranatrolite, edingtonite, kalborsite, analcime, leucite, pollucite, wairakite, Laumontite (LAU), yugawaralite (YUG), goosecreekite (GOO), montesommaite (MON), harmotome, phillipsite, amicite, gismondine, garronite, gobbinsite, or a synthetic zeolite, preferably Zeolite A, or combinations thereof.

34. The method of any one of claims 21-33, wherein said chemicals are renewable fuels, chemicals and materials, preferably ethanol, butanol, lactic acid, gasoline, biodiesel, methane, hydrogen, electricity, plastics, composites, protein, drugs, fertilizers, or combinations thereof.

35. The method of any one of claims 21-33, wherein said chemicals are succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

36. The method of claim 35, wherein said chemicals are 5-hydroxymethyl furfural (HMF), furfural, 2,5-furandicarboxylic acid, formic acid, levulinic acid, or mixtures thereof.

37. The method of claim any one of claims 1-36, wherein said ionic liquid is molten at a temperature ranging from about 10° C. to 160° C. and comprises cations or anions.

38. The method of claim 37, wherein the ionic liquid comprises a cation structure that includes ammonium, sulfonium, phosphonium, lithium, imidazolium, pyridinium, picolinium, pyrrolidinium, thiazolium, triazolium, oxazolium, or combinations thereof.

39. The method of claim 37, wherein the ionic liquid comprises a cation selected from imidazolium, pyrrolidinium, pyridinium, phosphonium, ammonium, or a combination thereof.

40. The method of any one of claims 1-39, wherein the ionic liquid (IL) is 1-n-butyl-3-methylimidazolium chloride, 1-allyl-3-methyl imidazolium chloride, 3-methyl-N-butylpyridinium chloride, 1-ethyl-3-methyl imidazolium acetate, 1-ethyl-3-methyl imidazolium propionatem, or combinations thereof.

41. The method of claim any one of claims 1-39, wherein said ionic liquid is 1-Butyl-3-methylimidazolium alkylbenzenesulfonate, 1-Ethyl-3-methylimidazolium alkylbenzenesulfonate, 1-Butyl-3-methylimidazolium acesulfamate, 1-Ethyl-3-methylimidazolium acesulfamate, 1-Ethylpyridinium chloride, 1-Butylimidazolium hydrogen sulfate, 1-Butyl-3-methylimidazolium hydrogen sulfate, 1-Butyl-3-methylimidazolium methyl sulfate, 1,3-Dimethylimidazolium methyl sulfate, 1-Butyl-3-methylimidazolium methanesulfonate, 1-Ethyl-3-methylimidazolium acetate, 1-Butyl-3-methylimidazolium acetate, 1-Octyl-3-methylimidazolium acetate, 1-(2-(2-Hydroxy-ethoxy)ethyl)-imidazolium acetate, 1-(2-(2-Methoxy-ethoxy)ethyl)-3-ethylimidazolium acetate, 1-(3,6,9,12-Tetraoxatridec-1-yl)-3-ethylimidazolium acetate, 1-(3,6,9,12,15,18,21-Heptaoxadocos-1-yl)-3-ethylimidazolium acetate, 1-(2-(2-Methoxy-ethoxy)ethyl)-triethylammonium acetate, (2-Hydroxy-ethyl)-dimethylammonium acetate, (2-Methoxyethyl)-dimethylammonium acetate, Tetramethylguanidinium acetate, Tetramethylguanidinium propionate, 1-Butyl-3-methylimidazolium formate, Tetrabutylphosphonium formate, Tetrabutylammonium formate, 1-Hexyl-3-methylimidazolium trifluoromethanesulfonate, 1-Butyl-3-methylimidazolium chloride, 1-Ethyl-3-methylimidazolium chloride, 1-Butyl-3-methylimidazolium bromide, 1-Allyl-3-methylimidazolium chloride, 1-Butyl-2,3-dimethylimidazolium tetrafluoroborate, 1-Butyl-3-methylimidazolium tetrafluoroborate, 1-Butyl-3-methylimidazolium hexafluorophosphate, 1-Butyl-1-methylpyrrolidinium hexafluorophosphate, 1-Ethyl-3-methylimidazolium diethyl phosphate, 1-Ethyl-3-methylimidazolium nitrate, 1,3-Dimethylimidazolium dimethyl phosphate, 1-Butyl-3-methylimidazolium dimethyl phosphate, 1-Methylimidazolium chloride, or combinations thereof.

42. The method of any one of claims 1-39, wherein the IL is represented by the structure: 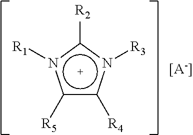
wherein each of R1, R2, R3, R4, and R5 is hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

43. The method of any one of claims 1-39, wherein the IL is represented by the structure: 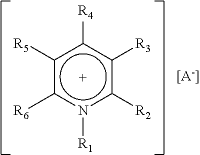
wherein each of R1, R2, R3, R4, R5, and R6 is hydrogen, an alkyl group having 1 to 15 carbon atoms or an alkene group having 2 to 10 carbon atoms, wherein the alkyl group may be substituted with sulfone, sulfoxide, thioether, ether, amide, hydroxyl, or amine and wherein A is a halide, hydroxide, formate, acetate, propanoate, butyrate, any functionalized mono- or di-carboxylic acid having up to a total of 10 carbon atoms, succinate, lactate, aspartate, oxalate, trichloroacetate, trifluoroacetate, dicyanamide, or carboxylate.

44. The method of claim 43 or 44, wherein the halide is a chloride, fluoride, bromide or iodide.

45. The method of any one of claims 1-39, wherein the ionic liquid is an ionic liquid mixture with a composition described by Equation 1:
$${\sum }_{n = 1}^{20} {{[ {C}^{+} ]}_{n} [ {A}^{-} ]}_{n} \qquad ( 1 )$$
C⁺ denotes the cation of the IL and A⁻ denotes the anionic component in Equation 1.

46. The method of any one of claims 1-45, wherein said method further comprises washing the treated biomass, preferably after the ionic liquid treatment and/or mild alkaline treatment.

47. The method of claim 46, wherein said washing comprises washing the biomass with a liquid non-solvent for cellulose that is miscible with water and the ionic liquid (IL).

48. The method of claim 47, wherein the liquid non-solvent used for washing is water, an alcohol, acetonitrile or a solvent which dissolves the IL and thereby extracts the IL from the biomass.

49. The method of claim 48, wherein the alcohol is ethanol, methanol, butanol, propanol, or mixtures thereof.

50. The method of any one of claims 1-49, wherein said wash is recovered and treated with RF heating infrared (IR) heating to dehydrate the ionic liquid.

51. The method of claim 50, wherein said ionic liquid is recovered from the liquid non-solvent by a method selected from one or more of activated charcoal treatment, distillation, membrane separation, electro-chemical separation techniques, sold-phase extraction liquid-liquid extraction, or a combination thereof.

52. The method of claim 51, wherein said ionic liquid is recovered from the liquid non-solvent by application of electromagnetic heating, preferably radiofrequency heating or infrared heating, that dehydrates the ionic liquid.

53. The method of claim 52, wherein the method further comprises reusing the recovered IL for treating more biomass, preferably wherein at least 90, 91, 92, 93, 94, 95, 96, 97, 98, or 99% of the IL is recovered.

54. The method of claim 52, wherein the ionic liquid has a water content not exceeding about 1, 2, 3, 4, 5, 6, 7, 8, 9, 10, 11, 12, 13, 14, 15, 16, 17, 18, 19, 20, 21, 22, 23, 24, or 25%.

55. The method of any one of claims 1-54, wherein the biomass is subjected to additional heating with agitation, ultrasonic heating, electromagnetic (EM) heating, radiofrequency (RF) heating, convective heating, conductive heating, microwave irradiation, or a combination thereof, preferably during step (a), step (b), acid hydrolysis, or enzymatic hydrolysis.

56. The method of claim 55, wherein said additional heating comprises intermittent agitation during heating.

57. The method of claim 55, wherein said electromagnetic (EM) heating is radiofrequency (RF) heating or infrared (IR) heating.

58. The method of claim 55, wherein said electromagnetic energy is applied at a power of 100-1000 W, 1 KW-10 KW, or 5 KW-1 MW.

59. The method of claim 55, wherein said radiofrequency comprises a frequency between about 1-900 MHz, 300 kHz-3 MHz, 3-30 MHz, 30-300 MHz, 13, 13.56, 27, 27.12, 40, or 40.68 MHz.

60. The method of claim 55, wherein the infrared radiation is at a frequency range of about 430 THz down to 300 GHz.

61. The method of claim 55, wherein the infrared radiation is near-infrared (near IR) wavelengths at about 0.75-1.4 μm, mid-infrared (mid IR) wavelengths at about 3-8 μm, or far infrared (far IR) wavelengths at about 15-1,000 μm.

62. The method of claim 55, wherein said radiofrequency heating penetrates the biomass to about 0.001 to 2.0 meters thickness.

63. The method of claim 55, wherein said infrared heating penetrates the biomass to about 0.001 to 2.0 meters thickness.

64. The method of claim 55, wherein heating comprises at least two phases, a first phase comprising application of electromagnetic (EM) heating, preferably a variable frequency in the electromagnetic spectrum, variable frequency heating, infrared (IR) heating, variable (IR) heating, radiofrequency (RF) heating, or a combination thereof, and a second phase comprising application of ultrasonics, electromagnetic (EM), convective, conductive heating, or combinations thereof.

65. The method of any one of claims 1-64, wherein said biomass is heated to a temperature of at least about 50-200° C., 80° C.-240° C., 50° C.-100° C., 60° C.-130° C., 80° C.-175° C., 100° C.-240° C., 100° C.-200° C., 90° C., 100° C., 105° C., 110° C., 115° C., 120° C., 125° C., 130° C., 135° C., 140° C., 145° C., or 150° C.

66. The method of any one of claims 1-65, wherein said biomass is heated for at least about 1 minute to 100 hours, 5 minutes to 8 hours, 3-30 minutes, 5-30 minutes, 3-4 hours, least 5-10 seconds, 1-30 minutes, 5-30 minutes, 1-360 minutes, 20-240 minutes, 1-60 minutes, 1-24 hours, 5-10 minutes, 5-30 minutes, 10-50 minutes, 5 minutes to 3 hours, 1-3 hours, 2-4 hours, 3-6 hours, or 4-8 hours.

67. The method of any one of the preceding claims, wherein said biomass is washed after step (a) and before step (b).

68. The method of any one of the preceding claims, wherein the lignin, cellulose, and/or hemicellulose is recovered.

69. The method of any one of the preceding claims, wherein the cellulose and hemicellulose are separated prior to hydrolysis.

70. The method of any one of the preceding claims, wherein said method further comprises processing the lignin to produce chemicals, binders, plastics, fuels, or combinations thereof.

71. The method of any one of the preceding claims, wherein the biomass is not dissolved in the ionic liquid.

72. The method of any one of the preceding claims, wherein the incubating step comprises incubating the biomass for a time ranging from about 5 minutes to about 8 hours.

73. The method of any one of the preceding claims, wherein the incubating step comprises incubating the biomass at a temperature ranging from about 50° C. to about 200° C.

74. The method of any one of the preceding claims, wherein the cellulose and hemicellulose structure in the biomass is swollen at least about 10%, 20%, 30%, or 40% by volume compared to before the ionic liquid incubation step.

75. The method of any one of the preceding claims, wherein the biomass is subjected to heating with agitation, ultrasonics heating, electromagnetic (EM) heating, preferably radiofrequency heating or infrared heating, convective heating, conductive heating, microwave irradiation, or a combination thereof, preferably with intermittent agitation during heating.

76. The method of any one of the preceding claims, wherein said biomass is agricultural residues, preferably corn stover, wheat straw, bagasse, rice hulls, or rice straw; wood and forest residues, preferably pine, poplar, douglas fir, oak, saw dust, paper/pulp waste, or wood fiber; algae; kudzu; coal; cellulose, lignin, herbaceous energy crops, preferably switchgrass, reed canary grass, or miscanthus; lingocellulosic biomass, preferably comprising lignin, cellulose, and hemicellulose; plant biomass; or mixtures thereof.

77. The method of claim 76, wherein said lignocellulosic biomass is agricultural residue, wood and forest residue, kudzu, herbaceous energy crop, lingocellulosic biomass comprising lignin, cellulose, and hemicellulose, plant biomass, or mixtures thereof.

78. The method of any one of the preceding claims, wherein said method is a continuous process.

79. The method of any one of the preceding claims, wherein said method is a batch process.

80. The method of claim 79, wherein said method is a fed-batch process.

81. The method of any one of the preceding claims, wherein said method comprises adjusting the amount of ionic liquid, the time of incubation, the pH of the biomass, and the temperature of the biomass.

82. The method of any one of the preceding claims, wherein the conditions of said biomass undergoing treatment is monitored with sensors, preferably a liquid flow rate sensor, thermocouple sensor, temperature sensor, salinity sensor, or combinations thereof.

83. The method of any one of the preceding claims, wherein the hemicellulose, cellulose, and/or lignin is separated.

84. The method of any one of the preceding claims, wherein the hemicellulose, cellulose, and/or lignin are converted to fuels, chemicals, polymers, or mixtures thereof.

85. The method of any one of the preceding claims, wherein the method does not comprise the use of a cellulase or hemicellulase.

86. A method for conversion of the carbohydrates of lignocellulosic biomass to sugars comprising
(a) mixing lignocellulosic biomass in an ionic liquid (IL) to swell but not dissolve the biomass;
(b) applying radio frequency (RF) heating to the lignocellulosic biomass to heat to a target temperature range;
(c) applying ultrasonics, electromagnetic (EM), convective, conductive heating, or combinations thereof, to the lignocellulosic biomass to maintain the lignocellulosic biomass at said target temperature range of about 50-220° C.;
(d) washing the treated lignocellulosic biomass;
(e) subjecting said lignocellulosic biomass to mild alkaline treatment to release lignin from the cellulosic components;
(f) washing the treated lignocellulosic biomass;
(g) recovering the lignin, cellulose, and hemicellulose; and
(h) hydrolyzing the cellulose and hemicellulose to yield sugars.

87. The method of claim 86, wherein said hydrolyzing comprises acid hydrolysis or enzyme hydrolysis.

88. A method for treatment of lignocellulosic biomass comprising
(a) incubating a biomass in a sufficient amount of an ionic liquid (IL) for a sufficient time and temperature to swell the lignocellulosic biomass without dissolution of the lignocellulosic biomass in the IL;
(b) applying radio frequency (RF) heating to the lignocellulosic biomass to heat to a target temperature range;
(c) applying ultrasonic heating to the lignocellulosic biomass to maintain the biomass at said target temperature range;
(d) washing the pretreated lignocellulosic biomass;
(e) subjecting said lignocellulosic biomass to mild alkaline treatment to release lignin from the cellulosic components;
(f) washing the treated lignocellulosic biomass with a liquid non-solvent for cellulose that is miscible with water and the IL;
(g) recovering the lignin, cellulose, and hemicellulose; and
(h) contacting said washed treated lignocellulosic biomass with an aqueous buffer comprising enzymes capable of hydrolyzing cellulose and hemicellulose to produce sugars.

89. A method of acidic hydrolysis of biomass comprising
(a) reducing the biomass in size, preferably to particles about 0.1-20 mm in size;
(b) incubating a biomass in a sufficient amount of an ionic liquid (IL) for a sufficient time and temperature to swell the lignocellulosic biomass without dissolution of the lignocellulosic biomass in the IL;
(c) treating the biomass uder mild alkaline treatment to separate the lignin from the cellulose and hemicellulose;
(d) separating the cellulosic, hemicellulosic, and lignin streams;
(e) recovering the lignin;
(f) adding an acid to each of the cellulosic and hemicellulosic streams to lower the pH below pH 7, preferably adding a catalyst;
(g) heating the cellulosic and hemicellulosic streams to heat to a target temperature range, preferably about 150-200° C. for about 15-360 minutes; and
(h) recovering chemicals.

90. A method for conversion of the carbohydrates of lignocellulosic biomass to sugars comprising
(a) mixing lignocellulosic biomass in an ionic liquid (IL) to swell but not dissolve the biomass;
(b) applying ultrasonics, electromagnetic (EM), preferably radio frequency (RF), convective, conductive heating, or combinations thereof, to the lignocellulosic biomass to heat the lignocellulosic biomass at a temperature range of about 50-220° C.;
(c) washing the treated lignocellulosic biomass;
(d) subjecting said lignocellulosic biomass to mild alkaline treatment to release lignin from the cellulosic components;
(e) washing the treated lignocellulosic biomass;
(f) recovering the lignin, cellulose, and hemicellulose;
(g) separating the lignin, cellulose, and hemicellulose;
(h) hydrolyzing the cellulose to yield sugars; and
(i) hydrolyzing the hemicellulose to yield sugars.

91. A method for treatment of lignocellulosic biomass comprising
(a) incubating a biomass in a sufficient amount of an ionic liquid (IL) for a sufficient time and temperature to swell the lignocellulosic biomass without dissolution of the lignocellulosic biomass in the IL;
(b) applying radio frequency (RF) heating, ultrasonic heating, or a combination to the lignocellulosic biomass to maintain at a target temperature range;
(c) washing the pretreated lignocellulosic biomass;
(d) subjecting said lignocellulosic biomass to mild alkaline treatment to release lignin from the cellulosic components;
(e) washing the treated lignocellulosic biomass with a liquid non-solvent for cellulose that is miscible with water and the IL;
(f) recovering the lignin, cellulose, and hemicellulose;
(g) separating the lignin, cellulose, and hemicellulose;
(h) hydrolyzing the cellulose to yield sugars; and
(i) hydrolyzing the hemicellulose to yield sugars.

92. The method of any one of claims 86-91, wherein said hydrolyzing comprises acid hydrolysis or enzyme hydrolysis.

93. The method of any one of claims 86-91, wherein said sugars are processed by acid hydrolysis, preferably with a catalyst, to produce chemicals.

94. A method of acidic hydrolysis of biomass comprising
(a) reducing the biomass in size, preferably to particles about 0.1-20 mm in size;
(b) incubating a biomass in a sufficient amount of an ionic liquid (IL) for a sufficient time and temperature to swell the lignocellulosic biomass without dissolution of the lignocellulosic biomass in the IL;
(c) treating the biomass uder mild alkaline treatment to separate the lignin from the cellulose and hemicellulose;
(d) separating the cellulosic, hemicellulosic, and lignin streams;
(e) recovering the lignin;
(f) adding an acid to the cellulosic stream to lower the pH below pH 7, preferably adding a catalyst;
(g) adding an acid to the hemicellulosic stream to lower the pH below pH 7, preferably adding a catalyst;
(h) heating the cellulosic stream to heat to a target temperature range, preferably about 150-200° C. for about 15-360 minutes;
(i) heating the hemicellulosic stream to heat to a target temperature range, preferably about 150-200° C. for about 15-360 minutes; and
(j) recovering chemicals.

95. A method of acidic hydrolysis of biomass comprising
(a) reducing the biomass in size, preferably to particles about 0.1-20 mm in size;
(b) incubating a biomass in a sufficient amount of an ionic liquid (IL) for a sufficient time and temperature to swell the lignocellulosic biomass without dissolution of the lignocellulosic biomass in the IL;
(c) separating the cellulosic, hemicellulosic, and lignin streams;
(d) recovering the lignin;
(e) adding an acid to the cellulosic stream to lower the pH below pH 7, preferably adding a catalyst;
(f) adding an acid to the hemicellulosic stream to lower the pH below pH 7, preferably adding a catalyst;
(g) heating the cellulosic stream to heat to a target temperature range, preferably about 150-200° C. for about 15-360 minutes;
(h) heating the hemicellulosic stream to heat to a target temperature range, preferably about 150-200° C. for about 15-360 minutes; and
(i) recovering chemicals.

96. The method of any one of claims 93-95, wherein said chemicals are succinic acid, glycerol, 3-hydropropoionic acid, 2,5-dimethylfuran (DMF), 5-hydroxymethyl furfural (HMF), furfural, 2,5-furandicarboxylic acid, itaconic acid, levulinic acid, aldehydes, alcohols, amines, terephthalic acid, hexamethylenediamine, isoprene, polyhydroxyalkanoates, 1,3-propanediol, or mixtures thereof.

97. The method of claim 96, wherein said chemicals are 5-hydroxymethyl furfural (HMF), furfural, 2,5-furandicarboxylic acid, formic acid, levulinic acid, or mixtures thereof.
